(19) United States
(12) Patent Application Publication
(10) Pub. No.: US 2010/0119506 A1
(43) Pub. Date: May 13, 2010

(54) Effectors of PAR-2 Activation and Their Use in the Modulation of Inflammation

(75) Inventors: Tobias LITZENBURGER, Newtown, CT (US); Sandra MILLER, Muenchen (DE); Catrin PRACHT, Neuried (DE); Rainer BOXHAMMER, Muenchen (DE); Jeanne MAGRAM, Croton-on-Hudson, NY (US); Patricia GIBLIN, Brookfield, CT (US); Daniel RAJOTTE, Lorraine (CA)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/512,251

(22) Filed: Jul. 30, 2009

Related U.S. Application Data
(60) Provisional application No. 61/086,282, filed on Aug. 5, 2008.

(51) Int. Cl. A61K 39/395; C07K 16/00; A61P 37/06

(52) U.S. Cl. 424/133.1; 530/387.1; 530/387.3; 424/130.1

(57) ABSTRACT

The present invention relates to the recognition that PAR-2 receptors amplify the inflammatory response and that effectors of PAR-2 activation can thus be used to modulate the inflammatory response and thereby impart therapeutic benefit to patients. The invention is particularly directed to the use of PAR-2 effectors in the treatment of inflammation and nociception (pain) caused by inflammation, cancer and injury. The invention is particularly directed to negative effectors of PAR-2 activation, and more particularly to anti-PAR-2 antibodies that are negative effectors of PAR-2 activation.

### CROSS-REFERENCE TO RELATED APPLICATIONS

This application claims priority to U.S. Provisional Patent Application No. 61/086,282, which was filed on Aug. 5, 2008, and which is incorporated by reference herein in its entirety.

### BACKGROUND OF THE INVENTION

The present invention relates to the recognition that PAR-2 receptors amplify the inflammatory response and that effectors of PAR-2 activation can thus be used to modulate the inflammatory response and thereby impart therapeutic benefit to patients. The invention is particularly directed to the use of PAR-2 effectors in the treatment of inflammation and nociception (pain) caused by inflammation, cancer and injury.

### I. PAR-2 and G-Protein Coupled Receptors

The G-Protein Coupled Receptors (“GPCRs”) comprise a large family of membrane proteins that share a common structural motif of seven hydrophobic transmembrane (“TM”) segments joined together by, an extracellular amino terminus and an intracellular carboxyl terminus (Kobilka, B. K. (epub 2006) Biochim. Biophys. Acta. 1768(4):794-807. GPCRs are the predominant mediators of transmembrane signaling in response to hormones, neurotransmitters, and sensory activation (e.g., sight, smell and taste). Multiple subfamilies of GPCRs (for example, the Rhodopsin subfamily, the Adhesion subfamily, the Frizzled/Taste subfamily, the Glutamate subfamily, the Secretin subfamily and the Protease-Activated Receptor (“PAR”) subfamily) have been identified through sequence analysis (Attwood, T. K. et al. (1994) Protein Eng. 7(2):195-203; Kolakowski, L. F., Jr. (1994) Receptors Channels 2(1):1-7; Fredriksson, R. et al. (2003) Mol. Pharmacol. 63(6):1256-1272; Macfarlane, S. R. et al. (2001) Pharmacol. Rev. 53(2):245-282). Despite the structural similarity of GPCRs, different GPCRs exhibit different functions and bind to structurally diverse natural ligands (Kobilka, supra; Ji, T. H. et al. (1998) J. Biol. Chem. 273(28):17299-17302).

Four members of the PAR subfamily of G-protein coupled receptors have been described: PAR-1, PAR-2, PAR-3 and PAR-4 (Hollenberg, M. D. et al. (2004) Br. J. Pharmacol. 143(4):443-454; Hansen, K. K. et al. (2004) Immunol. 112 (2):183-190; Hollenberg, M. D. (2003) Life Sci. 74(2-3):237-246; Hollenberg, M. D. (2000) Mol. Pharmacol. 58(6):1175-1177; Hollenberg, M. D. et al. (2000) Can. J. Physiol. Pharmacol. 78(1):81-85; Hollenberg, M. D. (1999) Trends Pharmacol Sci. 20(7):271-273; Hollenberg, M. D. et al. (1999) Can. J. Physiol. Pharmacol. 77(6):458-464).

PAR subfamily members are characterized by a unique mode of activation. Rather than being stimulated by the binding of a receptor ligand as is the case with other GPCRs, PAR subfamily members are activated enzymatically through the proteolytic cleavage of a portion of their N-terminal region. This cleavage, which is mediated by a serine protease, enables the remaining portion of the N-terminal domain of the receptor to adopt a conformation that allows it to bind to residues within the second extracellular loop of the PAR molecule. Such binding mediates the activation of the receptor. Because the post-proteolytic portion of the N-terminal domain is part of the receptor molecule, it is referred to as a “tethered” ligand (see, Macfarlane et al., supra). The predominant proteases known to be responsible for cleaving PAR-2 are tryptase and trypsin.

The activation of PAR receptors induces multiple cellular, tissue and systemic effects. PAR-2 is of particular relevance to the present invention, and is discussed in U.S. Pat. Nos. 7,256,172; 7,148,018; 6,864,229 and 6,323,219; U.S. Patent Publication Nos. 20070237759; 20060142203; 20060063930; 20040082773; 20040266687; 20020045581; European Patent Nos. 1511765 and 1238283; PCT Publication Nos. WO 07/092,640; WO 07/076,055; WO 06/127396; WO 06/127379; WO 06/104190; WO 06/070780; WO 06/035936; WO 06/023844; WO 04/003202; WO 04/002418; and WO 01/52883; and in Hansen et al., supra; Hollenberg (2003), supra; Hollenberg et al. (2000), supra; and Macfarlane et al., supra). PAR-2 activation has been found to induce responses in vascularized tissue and highly vascularized organs (Nystedt, S. et al. (1995) Eur. J. Biochem. 232(1):84-89; Nystedt, S. et al. (1994) Proc. Natl. Acad. Sci. (U.S.A.) 91:9208-9212). Studies have shown it to affect broncheorelaxation, calcium uptake by osteoblasts, saliva production, mitogenesis, as well as to affect the gallbladder, intestine, kidneys, pancreas, nervous system, immune system, stomach and ureter. PAR-2 activation mediates multiple effects on keratinocytes: increasing calcium uptake, inhibiting proliferation and differentiation, inducing IL-6 and GM-CSF production, increasing pigmentation, increasing melanin uptake and increasing phagocytosis (Derian, C. K. et al. (1997) Cell Growth Differen. 8:743-749; Santulli, R. J. et al. (1995) Proc. Natl. Acad. Sci. (U.S.A.) 92:9151-9155; Seiberg, M. et al. (2000) Exp. Cell Res. 254:25-32; Seiberg, M. et al. (2000) J. Invest. Dermatol. 115:162-167; Sharlow, E. R. et al. (2000) J. Cell. Sci. 113:3093-3101). Macfarlane et al., supra, reviews the numerous effects of PAR activation.

### II. Inflammation

The term “inflammation” is employed to describe a complex response of the immune system aimed at achieving the accumulation and activation of leukocytes and plasma proteins at sites of infection, toxin exposure or cellular injury in order to remove the injurious stimuli and initiate the process of repair/healing (Abbas, A. K. et al. (2000) CELLULAR AND MOLECULAR IMMUNOLOGY, 4^{th} Ed., W.B. Saunders, Philadelphia, Pa.). While the inflammatory response is thus often desirable, aberrant regulation of the immune response can cause the initiation of an inappropriate inflammatory response, which, if untreated, can lead to pain and tissue damage (see, e.g., Hickey, PSYCHONEUROIMMUNOLOGY II (Academic Press 1990).

Steroidal anti-inflammatory drugs (such as the glucocorticoids: hydrocortisone, prednisone, methylprednisone, dexamethasone, betamethesone, and fludrocortisone) are among the most potent, rapid, and reliable anti inflammatory agents available. These agents act to suppress the two main products in inflammation: prostaglandins and leukotrienes. Unfortunately, the use of existing steroidal anti-inflammatory drugs can be attended by significant adverse side effects, including: general immunosuppression, hyperglycemia, increased skin fragility, reduced bone density (osteoporosis, higher fracture risk, slower fracture repair), weight gain, muscle breakdown (proteolysis), weakness, reduced muscle mass, growth failure, pubertal delay and central nervous system excitation (see, e.g., U.S. Patent Publication No. 20070275938; van Staa, T. P. (2006) Calcif. Tissue Int. 79(3):129-137; Goodman, S. B. et al. (2007) J. Am. Acad. Orthop. Surg. 15(8):450-460; Irving, P. M. et al. (2007) Aliment Pharmacol Ther. 26(3):313-329; Devogelaer, J. P. (2006) Rheum. Dis. Clin. North Am. 32(4):733-757; Allen, D. B. (2006) Adv. Pediatr. 53:101-110; El Desoky, E. S. (2001) Curr. Therap. Res. 62(2):92-112).

Non-steroidal anti-inflammatory drugs (NSAIDs) comprise the most widely used class of therapeutic agents (see, e.g., Antman, E. M. et al. (Epub Feb. 26, 2007) Circulation 115(12):1634-1642; Adachi, M. et al. (2007) Histol. Histopathol. 22(4):437-442; Fortun, P. J. et al. (2007) Curr. Opin. Gastroenterol. 23(2):134-141; Gaffo, A. et al. (2006) Amer. J. Health Syst. Pharm. 63(24):2451-2465; Rosen, J. et al. (2005) Endocr. Rev. 26(3):452-464). Although these drugs all exhibit analgesic, anti-inflammatory and antipyretic properties by inhibiting expression of the cyclo-oxygenase-2 (COX-2) isoform (Grosser, T. (2006) Thromb. Haemost. 96(4):393-400; Hawkey, C. J. et al. (2005) Curr. Opin. Gastroenterol. 21(6):660-664; Curry, S. L. et al. (2005) J. Amer. Anim. Hosp. Assoc. 41(5):298-309), they comprise a structurally diverse set of agents, including salicylic acid (aspirin), naproxen, ibuprofen, Vioxx®, Celebrex®, diclofenac-Na, meloxicam and nimesulide. Unfortunately, the use of NSAIDs is also attended by significant risk (U.S. Patent Publication Nos. 20070237740 and 20070184133). NSAIDs that are not COX-2 selective are reported to produce gastrointestinal side effects in between 20% and 40% of all individuals who take them (Steinmeyer, J. (2000) Arthritis Res. 2(5):379-385). Even COX-2 selective NSAIDs, however, carry the potential for serious side effects. Serious gastrointestinal bleeding is seen in 1-2% of patients. With continuous therapy 15-20% of patients will develop peptic ulcers (Singh, G. et al. (1996) Arch. Intern. Med. 156:1530-1536; Brooks, P. M. et al. (1991) N. Engl. J. Med. 324:1716-1725).

Thus, despite all prior advances, a need remains for compositions that can be used to provide therapeutic or prophylactic treatment for inflammation and nociception (pain) caused by inflammation, cancer and other injury. The present invention relates to this and related needs.

### SUMMARY OF THE INVENTION

The present invention relates to the recognition that PAR-2 receptors amplify the inflammatory response and that effectors of PAR-2 activation can thus be used to modulate the inflammatory response and thereby impart therapeutic benefit to patients. The invention is particularly directed to the use of PAR-2 effectors in the treatment of inflammation and nociception (pain) caused by inflammation, cancer and injury. The invention is particularly directed to negative effectors of PAR-2 activation, and more particularly to anti-PAR-2 antibodies that are negative effectors of PAR-2 activation.

In detail, the invention provides a composition comprising a Protease Receptor-2 (PAR-2) effector molecule comprising a domain capable of binding to a region of PAR-2, wherein the binding modulates the activation or activity of PAR-2. The invention particularly concerns the embodiment of such PAR-2 effector molecules wherein the molecule is a negative effector of PAR-2 activation or activity.

The invention particularly concerns the embodiment of such PAR-2 effector molecules, wherein the molecule is an antibody (and especially an engineered antibody). The invention particularly concerns the embodiment of such engineered antibodies wherein the engineered antibody exhibits attribute (A), (B) or (C), or any combination of attributes (A), (B) and (C):
(A) exhibiting a Kd of from about 0.03 nM to about 0.2 nM.;
(B) exhibiting a concentration-dependent binding to human PAR-2 transfected HEK293 cells with an EC50 of from about 0.2 nM to about 0.4 nM; and/or
(C) inhibiting trypsin-induced calcium release in an epithelial cell (and especially in an epithelial cell of a human, rat or mouse epithelial cell line) with an IC50 of from about 2.0 nM to about 0.5 nM, and more preferably with an IC50 of from about 1.0 nM to about 0.5 nM.

The invention particularly concerns the embodiment of such PAR-2 effector molecules wherein the effector molecule is an engineered antibody that exhibits at least 100-fold greater binding affinity to human PAR-2 relative to human PAR-1.

The invention particularly concerns the embodiment of such PAR-2 effector molecules wherein the effector molecule is an engineered antibody that comprises a heavy chain CDR having the sequence of any of SEQ ID NOs:11-15 or 17 or a light chain CDR having the sequence of any of SEQ ID NOs:19-20. The invention particularly concerns the embodiment of such PAR-2 effector molecules wherein the effector molecule is an engineered antibody that comprises a heavy chain CDR having the sequence of any of SEQ ID NOs:11-15 or 17 and a light chain CDR having the sequence of SEQ ID NO:19. The invention particularly concerns the embodiment of such PAR-2 effector molecules wherein the effector molecule is an engineered antibody that comprises a heavy chain CDR having the sequence of any of SEQ ID NOs:11-15 or 17 and a light chain CDR having the sequence of SEQ ID NO:20.

The invention particularly concerns the embodiment of such PAR-2 effector molecules wherein the effector molecule is an engineered antibody that comprises a heavy chain CDR having the sequence of any of SEQ ID NOs:21-26 or SEQ ID NOs:30-36 or a light chain CDR having the sequence of any of SEQ ID NOs:27-28. The invention particularly concerns the embodiment of such PAR-2 effector molecules wherein the effector molecule is an engineered antibody that comprises a heavy chain CDR having the sequence of any of SEQ ID NOs:21-26 or SEQ ID NOs:30-36 and a light chain CDR having the sequence of SEQ ID NO:27. The invention particularly concerns the embodiment of such PAR-2 effector molecules wherein the effector molecule is an engineered antibody that comprises a heavy chain CDR having the sequence of any of SEQ ID NOs:21-26 or SEQ ID NOs:30-36 and a light chain CDR having the sequence of SEQ ID NO:28.

The invention particularly concerns the embodiment of such PAR-2 effector molecules wherein the effector molecule is the engineered antibody Par-B, Par-C or Par-D. The invention particularly concerns the embodiment of such PAR-2 effector molecules wherein the effector molecule is an engineered antibody having an antibody light chain whose amino acid sequence is encoded by an expressed DNA sequence selected from the DNA sequences of SEQ ID NO:37 or SEQ ID NO:40 and/or having a heavy chain whose amino acid sequence is encoded by an expressed DNA sequence selected from the DNA sequences of SEQ ID NO:38, SEQ ID NO:39 or SEQ ID NO:41.

The invention also concerns a pharmaceutical composition comprising any of the above-described PAR-2 effector molecules and a pharmacologically acceptable excipient. The invention additionally concerns any of such pharmaceutical compositions that additionally comprises an additional anti-inflammatory agent.

The invention also provides a method for treating inflammation in a recipient mammal, comprising administering to the mammal a pharmaceutical composition, comprising any of the above-described PAR-2 effector molecules and a pharmacologically acceptable excipient, in an amount sufficient to provide such treatment. The invention additionally concerns any of such methods wherein the provided compositions additionally comprises an additional anti-inflammatory agent. The invention particularly concerns any of such methods wherein the inflammation is selected from the group consisting of: psoriasis, contact dermatitis, inflammatory bowel disease, trans vivo delayed type hypersensitivity, PAR-2 mediated aortic ring relaxation and pain.

The invention also provides a method for preventing or inhibiting inflammation in a recipient mammal, comprising administering to the mammal, in advance of the inflammation, a pharmaceutical composition, comprising any of the above-described PAR-2 effector molecules and a pharmacologically acceptable excipient, in an amount sufficient to provide such prevention or inhibition. The invention also provides the embodiment of such method wherein the composition additionally comprises an additional anti-inflammatory agent. The invention particularly concerns the embodiment of such methods wherein the inflammation is selected from the group consisting of: psoriasis, contact dermatitis, inflammatory bowel disease, trans vivo delayed type hypersensitivity, PAR-2 mediated aortic ring relaxation and pain.

### BRIEF DESCRIPTION OF THE FIGURES

FIG. 1, Panels A-C illustrate the structure of PAR-2 within the cellular membrane. FIG. 1, Panel A shows the general structure of PAR-2. FIG. 1, Panel B illustrates the activation of PAR-2 upon proteolytic cleavage of its N-terminal domain. Activation of receptors of the PAR family is mediated by proteolytic cleavage resulting in a new N-terminus. This newly exposed ligand then participates in an intramolecular interaction with the second extracellular loop of these GPCRs resulting in downstream signaling events. Small peptides matching the sequence of the tethered ligand can also activate individual PAR receptors and thus are used as important tools for target validation (FIG. 1, Panel C).

FIG. 2 shows that antibody Ab3777 exhibits partial inhibition of trypsin cleavage of PAR-2 in a FRET-based peptide cleavage assay.

FIG. 3 shows that antibody Ab3777 exhibits partial inhibition in a cellular cleavage assay. The Legend to FIG. 3 is shown in Table 1.

FIG. 4 shows the results of a study of the binding of affinity matured antibodies IgG4_Pro Ab4996, Ab4999, Ab5005 and the parental IgG1 Ab3777 to PAR-2 transfected HEK293 cells.

FIG. 5 shows representative data of a FLAG-PAR-2 cleavage assay testing affinity-optimized IgGs. Detection of the FLAG-tag on the cell surface correlates with inhibitory activity.

FIG. 6 shows that Antibody Ab3777 exhibits partial inhibition in a trypsin induced calcium response assay.

FIG. 7 shows the potent inhibition of trypsin induced relaxation of rat aortic rings ex vivo and demonstrates that anti-PAR-2 antibodies of the present invention can functionally inhibit a biological response mediated through PAR-2 activation.

FIGS. 8A and 8B show the ability of anti-PAR-2 antibodies Par-B (FIG. 8A) and Par-D (FIG. 8B) to inhibit trans vivo DTH. Shown are the changes in paw thickness after injection of human peripheral blood mononuclear cells alone (PBMC) or after injection of PBMCs and tentanus toxin (TT). Mice were pretreated (ip at −3 hours) with PBS, hu IgG4 (20 mg/kg), or anti-PAR-2 antibody. Percent inhibition is shown at top of the bars. *p<0.05 as compared to PBS.

FIG. 9 depicts the concentration-dependent inhibition of IL-8 secretion by the anti-PAR-2 antibodies Par-B and Par-D.

### DETAILED DESCRIPTION

The role of PAR-2 in the inflammatory response has been well documented in a variety of settings in both rodents and humans (Cottrell, G. S. et al. (2003) Biochem. Soc. Trans. 31:1191-1197). Although PAR-2 receptors are expressed broadly at low levels, they are activated locally in response to inflammatory stimuli leading to cytokine production and cellular proliferation. Increased proteolytic activity and increased PAR-2 expression have been demonstrated at sites of inflammation in several different diseases. Early inflammatory signals that result in the degranulation of mast cells stimulate the release of tryptase and trypsin which act directly on the PAR-2 receptor. Stimulation of PAR-2 receptors amplifies the inflammatory response leading to cytokine production and cellular proliferation. PAR-2 has also been shown to play a role in nociception and neurogenic inflammation (see, Bunnett, N. W. (2006) Semin. Thromb. Hemost. 32 (Suppl. 1):39-48), and in cancer indications (see, O'Brien, P. J. et al. (2001) Oncogene 20:1570-1581).

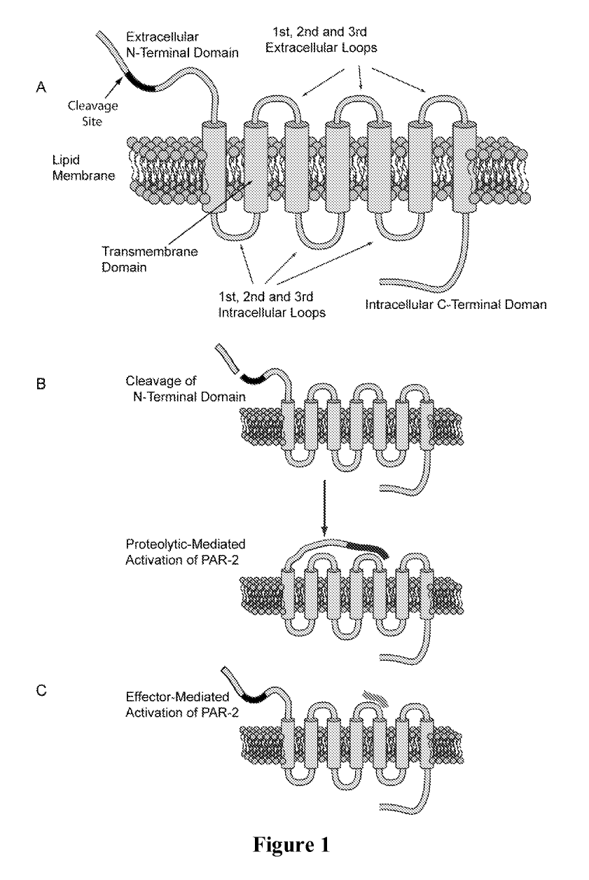

As discussed above, PAR-2 (FIG. 1, Panel A) is activated through the proteolytic cleavage of its N-terminus, which results in the creation of a “tethered” ligand capable of binding to residues of the second transmembrane loop to thereby activate the receptor (FIG. 1, Panel B).

The present invention derives, in part, from the recognition that PAR-2 receptors amplify the inflammatory response and that effectors of PAR-2 activation can thus be used to modulate the inflammatory response and thereby impart therapeutic benefit to patients. The invention is particularly directed to the use of PAR-2 effectors in the treatment of inflammation and nociception (pain) caused by inflammation, cancer and injury.

As used herein, an “effector” of PAR-2 activation is a molecule that modulates the activation or activity of PAR-2. Such effector molecules may be “positive” effectors of PAR-2 activation, in that they activate PAR-2 (or accelerate the activation of PAR-2). Alternatively, such molecules may be “negative” effectors of PAR-2, such that they inhibit or prevent PAR-2 activation (or attenuate the kinetics of PAR-2 activation). Thus the effectors of PAR-2 of the present invention include effectors that up-modulate PAR-2 activation so as to increase inflammation in a recipient, as well as effectors that down-modulate PAR-2 activation so as to decrease or prevent inflammation in a recipient. Positive effectors of PAR-2 are desirable to augment an ineffective or insufficient immune response (e.g., in the case of, for example, bacterial, fungal or viral infection, cancer or aging) (see, Paludan, S. R. (2000) J. Leukocyte Biol. 67(1):18-25; Garbino, J. et al. (2007) Expert Rev. Anti. Infect. Ther. 5(1):129-140; Pugin, J. (2007) Novartis Found Symp. 280:21-27). Negative effectors of PAR-2 are desirable to prevent or inhibit undesired inflammation.

Without limitation to the invention, the PAR-2 effectors of the present invention may operate through any of a variety of mechanisms. For example, positive PAR-2 effectors of the present invention may act as mimetics of the tethered ligand and as such mediate activation of the receptor (FIG. 1, Panel C). Alternatively, PAR-2 effectors of the present invention may act to inhibit or prevent binding from occurring between the “tethered” ligand and the relevant residues of the PAR-2 second transmembrane loop, thereby inhibiting or preventing PAR-2 activation. PAR-2 effectors of the present invention may act to facilitate the cleavage of the PAR-2 N-terminus, thereby increasing the kinetics and extent of PAR-2 activation. Conversely, PAR-2 effectors of the present invention may act to prevent cleavage of the PAR-2 N-terminus, thereby attenuating or preventing PAR-2 activation. The ability of the PAR-2 effectors of the present invention can be itself modulated by increasing or decreasing the binding affinity of such effectors for their respective targets. The present invention particularly contemplates the therapeutic use of negative effectors of PAR-2 activation.

The term “IC₅₀” denotes the concentration of a drug that is required for 50% inhibition in vitro whereas the term “EC₅₀” denotes the plasma concentration required for obtaining 50% of a maximum effect in vivo.

The invention particularly concerns high efficacy PAR-2 effectors capable of in vivo prophylactic or therapeutic use in the treatment of inflammation. As used herein, a “high efficacy” PAR-2 effector is a PAR-2 effector having an EC₅₀ less than 6 nM, preferably, less than 2 nM, more preferably less than 1 nM, and still more preferably less than 0.5 nM, less than 0.2 nM, or less than 0.1 nM. Preferably, such high efficacy PAR-2 effectors will additionally exhibit an IC₅₀ of less than 6 nM, preferably, less than 2 nM, more preferably less than 1 nM, and still more preferably less than 0.5 nM, less than 0.2 nM, or less than 0.1 nM, and a dissociation constant (Kd) of less than 0.5 nM, less than 0.2 nM, less than 0.1 nM, or more preferably, less than 0.05 nM.

The term “inflammation,” as used herein, is meant to include conditions and diseases resulting from reactions of either the specific or non-specific defense systems. As used herein, the term “specific defense system” is intended to refer to that component of the immune system that reacts to the presence of specific antigens. Inflammation is said to result from a response of the specific defense system if the inflammation is caused by, mediated by, or associated with a reaction of the specific defense system. Examples of inflammation resulting from a response of the specific defense system include the response to antigens such as rubella virus, autoimmune diseases, delayed type hypersensitivity response mediated by T-cells (as seen, for example in individuals who test “positive” in the Mantaux test), etc. A “non-specific defense system reaction” is a response mediated by leukocytes incapable of immunological memory. Such cells include granulocytes and macrophages. As used herein, inflammation is said to result from a response of the non-specific defense system, if the inflammation is caused by, mediated by, or associated with a reaction of the non-specific defense system. Examples of inflammation which result, at least in part, from a reaction of the non-specific defense system include inflammation associated with conditions such as: asthma; adult respiratory distress syndrome (ARDS) or multiple organ injury syndromes secondary to septicemia or trauma; reperfusion injury of myocardial or other tissues; acute glomerulonephritis; psoriasis, rheumatoid arthritis; reactive arthritis; dermatoses with acute inflammatory components; contact dermatitis; acute purulent meningitis or other central nervous system inflammatory disorders; thermal injury; hemodialysis; leukapheresis; inflammatory bowel disease; ulcerative colitis; Crohn's disease; necrotizing enterocolitis; granulocyte transfusion associated syndromes; and cytokine-induced toxicity.

### I. Preferred Compositions

The invention particularly contemplates the use of antibody molecules as PAR-2 effectors. As used herein, the term “antibody” includes a polyclonal, monoclonal, bi-specific, multi-specific, human, humanized, or chimeric antibody, a single chain antibody, an antigen-binding fragment of an antibody (e.g., an Fab or F(ab′)₂ fragment), a disulfide-linked Fv, etc. Such antibodies may be produced through chemical synthesis (see, e.g., Dawson, P. E. et al. (2000) Ann. Rev Biochem. 69:923-960; Wilken, J. et al. (1998) Curr. Opin. Biotechnol. 9(4):412-426; Kochendoerfer, G. G. et al. (1999) Curr. Opin. Chem. Biol. 3(6):665-671), via recombinant or transgenic means (Wang, M. et al. (2007) IDrugs 10(8):562-565; Aubrey, N. et al. (2006) J. Soc. Biol. 200(4):345-354; Laffly, E., et al. (2006) J. Soc. Biol. 200(4):325-343; Hagemeyer, C. E. et al. (2007) Semin. Thromb. Hemost. 33(2):185-195; Rasmussen, S. K. et al. (2007) Biotechnol. Lett. 29(6):845-852; Gasser, B. et al. (2007) Biotechnol. Lett. 29(2):201-212; Jefferis, R. (2005) Biotechnol. Prog. 21(1):11-16; Smith, K. A. et al. (2004) J. Clin. Pathol. 57(9):912-917; Kipriyanov, S. M. et al. (2004) Mol. Biotechnol. 26(1):39-60; Fischer, R. et al. (2003) Vaccine 21(7-8):820-825; Maynard, J. et al. (2000) Ann. Rev. Biomed. Eng. 2:339-376; Young, M. W. et al. (1998) Res. Immunol. 149(6):609-610; Hudson, P. J. (1998) Curr. Opin. Biotechnol. 9(4):395-402), via cell (e.g., hybridoma) culture (Laffly et al., supra; Aldington, S. et al. (2007) J. Chromatogr. B Analyt. Technol. Biomed. Life Sci. 848(1):64-78; Farid, S. S. (2006) J. Chromatogr. B Analyt. Technol. Biomed. Life Sci. 848(1):8-18; Birch, J. R. et al. (2006) Adv. Drug Deliv. Rev. 58(5-6):671-685; Even, M. S. et al. (2006) Trends Biotechnol. 24(3):105-108; Graumann, K. et al. (2006) Biotechnol. J. 1(2):164-186; U.S. Pat. No. 7,112,439; U.S. Patent Publication Nos. 20070037216 and 20040197866), or by other means.

Most preferably, antibody PAR-2 effector molecules of the present invention will be isolated and/or refined (for example to improve their binding kinetics or specificity or to modulate such attributes) using bacteriophage display panning methods (see, e.g., U.S. Patent Publication No. 20070292947; PCT Publication Nos. WO 88/06630; WO 90/02809; WO 92/01047; WO 91/17271; WO 91/19818; WO 92/01047; and WO 92/09690; Parmley S. F. et al. (1988) Gene 73:305-318; Bass, S. et al. (1990) Proteins: Structure, Function and Genetics 8:309-314; Greenwood, J. et al. (1991) J. Mol. Biol. 220:821-827; Jespers L. S. et al. (1995) Biotechnology (N.Y.) 13:378-382; Kay, B. K. et al. (1996) In: PHAGE DISPLAY OF PEPTIDES AND PROTEINS: A LABORATORY MANUAL, Winter, J. et al. (Eds.) Academic Press, Inc., San Diego; Dunn, I. S. (1996) Curr. Opin. Biotechnol. 7:547-553; McGregor, D. (1996) Mol. Biotechnol. 6:155-162; Gao, C. et al. (1999) Proc. Natl. Acad. Sci. (U.S.A.) 96:6025-6030.

In one embodiment of the present invention, such PAR-2 effector antibodies will be selected for their ability to bind to an N-terminal PAR-2 peptide that spans the protease cleavage site for trypsin/tryptase. Such binding decreases the accessibility of this region to proteolytic cleavage and thus inhibits PAR-2 activation. Accordingly an antibody that immunospecifically binds to this region is a negative effector of PAR-2. As used herein, the term “immunospecifically binds,” refers to the specific binding that characterizes the interaction between an antibody and an antigen that elicits that antibody.

The PAR-2 N-terminal domain cleavage site (shown as “▾”) is located between positions 36-37 of the human PAR-2 protein (SEQ ID NO:1):

SEQ ID NO:1

(Human PAR-2; Genbank Accession No. P55085)
MRSPSAAWLL GAAILLAASL SCSGTIQGTN RSSKGR▾SLIG
KVDGTSHVTG KGVTVETVFS VDEFSASVLT GKLTTVFLPI
VYTIVFVVGL PSNGMALWVF LFRTKKKHPA VIYMANLALA
DLLSVIWFPL KIAYHIHGNN WIYGEALCNV LIGFFYGNMY
CSILFMTCLS VQRYWVIVNP MGHSRKKANI AIGISLAIWL
LILLVTIPLY VVKQTIFIPA LNITTCHDVL PEQLLVGDMF
NYFLSLAIGV FLFPAFLTAS AYVLMIRMLR SSAMDENSEK
KRKRAIKLIV TVLAMYLICF TPSNLLLVVH YFLIKSQGQS
HVYALYIVAL CLSTLNSCID PFVYYFVSHD FRDHAKNALL
CRSVRTVKQM QVSLTSKKHS RKSSSYSSSS TTVKTSY

Accordingly, peptide fragments of a suitable number of amino acid residues spanning the cleavage site are prepared and a library of candidate antibodies is screened against the peptide for binding. More specifically the synthetic HuCAL GOLD library (Ostendorp, R. et al. (2004) ANTIBODIES VOLUME 2: NOVEL TECHNOLOGIES AND THERAPEUTIC USE, pp. 13-52 Kluwer Academic/Plenum Publishers, New York; U.S. Pat. Nos. 5,514,548; 6,294,353; 6,300,064; 6,653,068; 6,667,150; 6,692,935; 6,696,248; 6,706,484; 6,753,136; 6,828,422; 7,049,135; 7,264,963) was used for selection of peptide specific binders. Antibodies that bind to the fragment are subjected to a affinity-maturation process (Brocks, B. et al. (2006) Hum. Antibodies 15 (4):115-124; Steidl, S. et al. www.priorartdatabase.com/IPCOM/000159278) to increase their affinity, using the above-described bacteriophage display techniques. The matured antibodies are screened against the peptide and PAR-2 expressing cells for increased binding.

Most preferably, the peptide fragment employed to identify antibody PAR-2 effector molecules will be a consensus sequence derived from a comparison of the sequences spanning the N-terminal cleavage sites of various mammalian PAR-2 molecules. For example, by comparing a 21 amino acid long peptide spanning the above-described N-terminal cleavage sites of human PAR-2 with the homologous sequences of murine (Genbank Accession No. CAE11955), rat (Genbank Accession No. NP_{—}446349), and cynomolgus monkey (Genbank Accession No. XP_{—}001106201) PAR-2, a preferred consensus sequence (SEQ ID NO:5) can be identified (conserved sequences are shown underlined):

SEQ ID NO: 1 (Residues 33-54)
SKGR▾SLIGKV DGTSHVTGKGV
SEQ ID NO: 2 (murine)
SKGR▾SLIGRL ETQPPITGKGV
SEQ ID NO: 3 (rat)
SKGR▾SLIGRL DTPPPITGKGA
SEQ ID NO: 4 (cynomolgus)
SKGR▾SLIGRV DGTFHVTGKGV
SEQ ID NO: 5 (Consensus)
SKGR▾SLIGRL DTTPPITGKG V

In a second embodiment of the present invention, such an antibody will be selected for its ability to bind the “tethered” ligand of the post-cleavage N-terminus of PAR-2. The sequence of the “tethered” ligand of the post-cleavage N-terminus of PAR-2 is present in SEQ ID NO: 1 (Residues 37-75): SLIGKVDGTS HVTGKGVTVE TVFSVDEFSA SVLTGKLTT. Antibodies that bind to the “tethered” ligand prevent or inhibit the ability of the “tethered” ligand to bind to residues of the second extracellular loop of PAR-2, and thus inhibit PAR-2 activation. Such molecules comprise negative effectors of PAR-2.

In a third embodiment of the present invention, such an antibody will be selected for its ability to bind to residues of the second extracellular loop of PAR-2. The sequence of the second extracellular loop of PAR-2 is found at SEQ ID NO: 1 (Residues 131-149): KIAYHIHGNN WIYGEALCN. Antibodies that bind to this region may cause partial or complete activation of PAR-2, for example by serving as a mimetic of the natural “tethered” ligand or by promoting binding of the natural “tethered” ligand to residues of the second extracellular loop of PAR-2, so as to comprise positive effectors of PAR-2. Alternatively, such antibodies may themselves be incapable of activating PAR-2 and may serve to inhibit the ability of the natural “tethered” ligand to mediate PAR-2 activation (and thus be negative PAR-2 effectors). Thus, antibodies that bind to the second extracellular loop of PAR-2 may be either positive effectors of PAR-2 or negative effectors of PAR-2, depending upon the nature of their interaction with the loop residues.

Most preferably, the antibody PAR-2 effector molecules will comprise “engineered antibodies.” As used herein, the term “engineered antibody” denotes that the antibody contains non-naturally-occurring sets of CDRs introduced into, for example, a naturally occurring IgG4 heavy chain and/or a λ light chain), and/or non-naturally occurring fusions of variable and constant chain regions (e.g., a fusion of a heavy chain variable region to the constant region of a different IgG (e.g., IgG2)). The invention particularly concerns engineered antibody that exhibit augmented binding relative to a pre-engineered or parental antibody. As used herein, an engineered antibody's binding is said to augmented if it is at least 5-fold, more preferably at least 10-fold, more preferably at least 20-fold, still more preferably at least 50-fold, still more preferably at least 100-fold, and most preferably at least 1000-fold greater than that exhibited by the pre-engineered or parental antibody.

Principles of codon optimization may be employed in order to adapt the polynucleotides encoding the antibody chains for augmented expression in different host cells as well as to modify RNA motifs which may interfere with expression. These changes to the nucleotide sequence do not lead to changes in the peptide sequence.

Although the preferred PAR-2 effector molecules of the present invention are antibodies, the present invention also encompasses non-antibody PAR-2 effector molecules. In one embodiment, such molecules will be non-antibody polypeptides, for example “antigen binding proteins” that bind to a PAR-2 epitope so as to effect PAR-2 activation. Alternatively, non-peptide PAR-2 effectors (for example, peptidomimetic compounds (see, e.g., US20070237759) may be employed. Generally, peptidomimetic compounds are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH2SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo, J. et al. (1992) Annu. Rev. Biochem. 61:387-418), for example, by adding internal cysteine residues capable of forming intramolecular disulfide bridges which cyclize the peptide.

The invention's provision of PAR-2 effectors permits the use of such effector molecules in screens for additional effector molecules. For example, a negative PAR-2 effector molecule (such as a PAR-2 effector antibody that immunospecifically binds an N-terminal PAR-2 peptide that spans the protease cleavage site for trypsin/tryptase) can be used with the FLIPR or other assays discussed below to permit the isolation of a molecule that reverses or augments the negative PAR-2 effect, thereby permitting the isolation of positive PAR-2 effector molecules or more potent negative PAR-2 effector molecules.

### II. Preferred Uses of the Compositions

Negative effectors of PAR-2 activation are of particular utility in the treatment of inflammation, such as in psoriasis, rheumatoid arthritis, contact dermatitis, inflammatory bowel disease, asthma. Psoriasis is an inflammatory disease of the skin that is characterized by lymphocyte infiltration, keratinoycte hyperproliferation and the production of pro-inflammatory cytokines and chemokines. Psoriasis is believed to result from an undesired inflammatory response that involves the elaboration of cytokines by abnormally activated T cells (Chong, B. F. et al. (2007) Clin. Immunol. 123(2):129-138; Ritchlin, C. (2007) Nat. Clin. Pract. Rheumatol. 3(12):698-706). The disease is reported to affect 1-3% of Caucasian populations and may be persistent, disfiguring and stigmatizing (MacDonald, A. et al. (2007) Postgrad. Med. J. 83(985):690-697; Sabat, R. et al. (2007) Exp. Dermatol. 16(10):779-798). No fully satisfactory therapy for the disease has yet been identified (Menter, A. et al. (2007) Lancet 370(9583):272-284.

Rheumatoid arthritis is an inflammatory disease characterized by joint inflammation, synovial hyperplasia and bone erosion. In the adjuvant arthritis model of rheumatoid arthritis, PAR-2 deficient mice had a significant reduction in joint swelling and no histological evidence of joint damage (Ferrell, W. R. et al. (2003) J. Clin. Invest. 111:35-41). Data of others suggest that the use of a small molecule inhibitor of PAR-2 (ENMD-1068) resulted in a decrease in clinical score in a carrageenan/kaolin-induced model of arthritis (Kelso, E. B. (2006) “Therapeutic Promise of Proteinase-Activated Receptor-2 Antagonism in Joint Inflammation,” J. Pharmacol. Exp. Ther. 316(3):1017-1024). Furthermore, intra-articular injection of a PAR-2 agonist is sufficient to induce prolonged joint swelling and synovial hyperemia. In the arthritic wild-type mice, PAR-2 expression was found to be upregulated in the synovium and surrounding periarticular tissues (Kelso, E. B. et al. (2006) J. Pharmacol. Exp. Ther. 316:1017-1024). However, another group has called into question these findings in PAR-2 knockout mice. Busso et al. failed to show inhibition of zymozan induced arthritis, K/BxN serum-induced arthritios and antigen-induced arthritis in PAR-2 knockout mice (See: Busso et al. (2007) Arthritis Rheumat. 56:101-107).

Contact dermatitis is an inflammatory response of skin to contact with external factors, such as irritants and allergens (Jacob, S. E. et al. (2007) Expert Opin. Pharmacother. 8(16):2757-2774; Diepgen, T. L. et al. (2007) J. Eur. Acad. Dermatol. Venereol. 21(Suppl 2):9-13; Brasch, J. et al. (2007) J. Dtsch. Dermatol. Ges. 5(10):943-951; Fyhrquist-Vanni, N. et al. Dermatol. Clin. 25(4):613-623; Amado, A. et al. (2007) Actas Dermosifiliogr. 98(7):452-458; Scalf, L. A. et al. (2007) Geriatrics 62(6):14-9).

Inflammatory Bowel Disease (IBD) is a chronic disorder characterized by recurrent and serious inflammation of the gastrointestinal tract. The disease has two main subforms, Crohn's disease and ulcerative colitis (Nikolaus, S. et al. (2007) Gastroenterology 133(5):1670-1689; Zhong, W. et al. (2008) Front Biosci. 13:1654-1664; Scaldaferri, F. et al. (2007) J. Dig. Dis. 8(4):171-178; Mitsuyama, K. et al. (2007) Anticancer Res. 27(6A):3749-3756; Yamamoto-Furusho, J. K. et al. (2007) World J. Gastroenterol. 13(42):5577-5580; He, S. H. (2004) World J. Gastroenterol. 10(3):309-318).

Asthma is a chronic inflammatory disorder of the airways. Asthma is thought to result from airway remodelling that is initiated and promoted by repeated episodes of allergic inflammation of the surface epithelium of the airway (Tang, M. L. K. et al. (2006) Pharmacol. Therap. 112(2):474-488; Caramori, G. et al. (2003) Pulmon. Pharmacol. Therap. 16(5):247-277; Kawabata, A. et al. (2005) J. Pharmacol. Sci. 97(1):20-24; Reed, C. E. et al. (2004) J. Allergy Clin. Immunol. 114(5):997-1009; Niu, Q. X. et al. (2003) Sheng Li Ke Xue Jin Zhan. 34(4):373-375; Black, J. L. (2002) Curr. Opin. Allergy Clin. Immunol. 2(1):47-51; Cocks, T. M. et al. (2001) Pulm. Pharmacol. Ther. 14(3):183-191). Despite advancements in the treatment of asthma, the disease continues to rise in prevalence, severity and mortality and is reported to affect approximately 10% of children and 5% of adults worldwide (Gupta, R. et al. (2004) Bioorg. Medicin. Chem. 12(24):6331-6342).

One aspect of the present invention relates to the recognition that PAR-2 activation plays a role in initiating the inflammatory cascade, and that therefore down-modulating effectors of PAR-2 activation may be used in the treatment of actual or anticipated cases of such inflammation. In this regard, inflamed tissue exhibits an increase in proteolytic activity that is accompanied by an increase in inflammatory cytokine production (e.g. IL-8) and granulocytic infiltration. PAR-2 provides a critical link between the early inflammatory response and the initial recruitment and activation of immune cells. PAR-2 expression and leukocyte recruitment are also increased in mouse contact sensitivity models. Consistent with the role of PAR-2 in, for example, the skin inflammation of psoriasis, PAR-2 deficient mice fail to mount a robust response in a model of contact hypersensitivity using oxazolone and picryl chloride (Kawagoe, J. et al. (2002) Jpn. J. Pharmacol. 88:77-84). In humans, increases in adhesion molecule expression (E-selectin and ICAM), mast cell activation and tryptase production are also observed following direct application of PAR-2 agonists to the skin of normal individuals (Seeliger, S. et al. (2003) FASEB J. 17:1871-1885). In patients with atopic dermatitis, however, injection of PAR-2 agonists induces an enhanced and prolonged itch (Steinhoff, M. et al. (2003) J. Neurosci. 23:6176-6180). In vitro, human keratinocytes secrete IL-8 in response to PAR-2 activation (Hou, L. et al. (1998) Immunology 94:356-362). Taken together these results support the conclusion of the present invention that PAR-2 plays a role in initiating the inflammatory cascade.

Negative effectors of PAR-2 activation are also of particular utility in the treatment of cancer (especially, blood cell cancer, brain cancer, breast cancer, colon cancer, head and neck cancer, liver cancer, lung cancer, ovarian cancer, pancreatic cancer, prostate cancer, skin cancer, and stomach cancer). Since PAR-2 participates in an autocrine loop that promotes proliferation, invasion, and metastasis, negative effectors of PAR-2 activation provide a therapy for cancer. (Soreide, K. et al. (2006) J. Pathol. 209(2):147-156; Versteeg, H. H. et al. (2006) Semin. Thromb. Hemost. 32(1):24-32; MacNaughton, W. K. (2005) Mem. Inst. Oswaldo Cruz. 100(Suppl 1):211-215; Nishibori, M. et al. (2005) J. Pharmacol. Sci. 97(1):25-30; Yamamoto, H. et al. (2003) Nippon Rinsho. 61(Suppl 7):220-224; Riewald, M. et al. (2002) Trends Cardiovasc. Med. 12(4):149-154; Leadley, R. J., Jr. et al. (2001) Curr. Opin. Pharmacol. 1(2):169-175; Macfarlane et al., supra).

The invention further contemplates the use of negative effectors of PAR-2 activation in the management of pain (nociception). Pain is initiated when noxious stimuli excite the peripheral terminals of specialized primary afferent neurons called nociceptors (see, Hwang, S. W. et al. (2007) Curr. Opin. Anaesthesiol. 20(5):427-434; Ma, W. et al. (2007) Expert Opin. Ther. Targets 11(3):307-320; Tominaga, M. (2007) Handbook Exp. Pharmacol. 179:489-505; Haddad, J. J. (2007) Biochem. Biophys. Res. Commun 353(2):217-224). Numerous molecules, including PAR-2 are involved in the amplification and propagation of nociception (Itoh, Y. et al. (2005) J. Pharmacol. Sci. 97(1):14-19; Coelho, A. M. et al. (2003) Curr. Med. Chem. Cardiovasc. Hematol. Agents 1(1):61-72; Vergnolle, N. et al. (2001) Trends Pharmacol. Sci. 22(3): 146-152).

The clinical significance of diseases such as immunodeficiency diseases (such as AIDS), cancer, and infection can be exacerbated by a failure to exhibit a suitable inflammatory response. In such cases, positive effectors of PAR-2 activation are of therapeutic benefit. For example, certain pathogens (e.g., Pseudomonas aeruginosa) have been shown to express proteases that cleave the N-terminal extracellular domain of PAR-2 at aberrant sites, thus affecting the ability of the resultant “tethered” ligand to bind to the residues of the extracellular loop and attenuating the host's inflammatory response (see, e.g., Chignard, M. et al. (2006) Am. J. Respir. Cell. Mol. Biol. 34(4):394-398). Other pathogens (e.g., E. coli, helminthes, viruses, etc.) also promote their survival in infected patients by inducing an anti-inflammatory environment (Magez, S. et al. (2006) J. Infect. Dis. 193(11):1575-158; Maizels, R. M. et al. (2004) Immunol. Rev. 201:89-116; Albee, L. (2006) Inflamm. Res. 55(1):2-9; Woodruff, J. F. (1979) J. Immunol. 123(1):31-36). Likewise, the innate immune system utilizes Toll-like receptors (TLRs) to recognize and bind pathogen-associated molecular patterns (PAMPs) leading to a proinflammatory and antibacterial response (e.g., defensin expression). Since PAR-2 activation also leads to defensin expression, positive PAR-2 effector molecules augment the desired antibacterial response in infections resulting from pathogens that avoid recognition by PAMPs (Froy, O. (2005) Cell. Microbiol. 7(10):1387-1397). Targeted inflammation has also been shown to be beneficial in treating cancer (Breitbach, C. J. et al. (Epub Jun. 19, 2007) Mol. Ther. 15(9):1686-1693).

### IIII. Administration of the Compositions

The invention contemplates the administration of the above-described effectors of PAR-2 to mammalian recipients, and in particular, to human recipients. The compositions of the present invention may be provided to such recipients in therapeutically effective amounts to modulate inflammation. As used herein, a “therapeutically effective amount” refers to that amount of a composition of the present invention sufficient to provide a desired modulation of inflammation in a recipient patient. The compositions of the present invention may be provided to recipients in therapeutically effective amounts to treat (enhance, in the case of positive effectors of PAR-2, or suppress, in the case of negative effectors of PAR-2) an ongoing inflammation in such recipient. As used herein, a “therapeutically effective amount” refers to that amount of a composition of the present invention sufficient to impart a therapeutic benefit in the treatment or management of such patient's inflammation. Alternatively, the compositions of the present invention may be provided to recipients in prophylactically effective amounts to prevent an undesired inflammation (negative effectors of PAR-2) or to induce inflammation in a patient likely to otherwise mediate an insufficient inflammatory response (positive effectors of PAR-2). As used herein, a “prophylactically effective amount” refers to that amount of a composition of the present invention sufficient to achieve such results upon administration to patients.

Therapeutic formulations of the compositions of the present invention may be prepared for storage by mixing a preparation of one or more effectors of PAR-2 having a desired degree of purity with optional physiologically acceptable-carriers, excipients or stabilizers (see, e.g., Gennaro, A. R. (2000) REMINGTON: THE SCIENCE AND PRACTICE OF PHARMACY, 20th Edition. Baltimore, Md.: Lippincott Williams & Wilkins)), in, for example, the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation may contain one or more than one species of PAR-2 effector molecules, alone or admixed with one or more than one non-PAR-2 inflammation modulators (collectively, “active compound(s)”) as necessary or desired to provide treatment for a particular indication. Preferably, such active compound(s) will exhibit complementary activities that do not adversely affect each other. Such active compound(s) are suitably present in combination in amounts that are effective for the purpose intended. If desired, such active compound(s) may be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in REMINGTON, supra. Formulations to be used for in vivo administration must be sterile. This is readily accomplished by filtration through sterile filtration membranes, or by other means well known in the art.

Sustained-release preparations may be prepared. Suitable examples of sustained-release preparations include semipermeable matrices of solid hydrophobic polymers containing the polypeptide variant, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers, and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a possible loss of biological activity and a possible change in immunogenicity. Rational strategies can be devised for stabilization depending on the mechanism involved. For example, if the aggregation mechanism is discovered to be intermolecular disulfide bond formation through thio-disulfide interchange, stabilization may be achieved by modifying sulfhydryl residues, lyophilizing from acidic solutions, controlling moisture content, using appropriate additives, and developing specific polymer matrix compositions.

Such pharmacological compositions of the present invention may be administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. Preferably the dosing is given by injections, most preferably intravenous or subcutaneous injections, depending in part on whether the administration is to be brief or chronic. The appropriate dosage of such pharmacological compositions will depend on the goal of the inflammation modulation, the severity and course of the disease or condition being treated, whether the pharmacological compositions are administered for preventive or therapeutic purposes, previous therapy, the patient's clinical history and prior response to the pharmacological compositions, and the discretion of the attending physician. The pharmacological compositions of the present invention are suitably administered to a patient at one time or over a series of treatments.

Depending on the type and severity of the disease, about 1 μg/kg to 15 mg/kg (e.g., 0.1-20 mg/kg) of the active compound(s) is an initial candidate dosage for administration to the patient, whether, for example, by one or more separate administrations, or by continuous infusion. A typical dosage (administered, for example once or twice per month) might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

Having now generally described the invention, the same will be more readily understood through reference to the following examples, which are provided by way of illustration and are not intended to be limiting of the present invention unless specified.

### Example 1

### Isolation and Properties of a PAR-2 Specific Antibody

Antibody phage display techniques were employed to pan for antibodies (Fab fragments) capable of immunospecifically binding to residues spanning the protease cleavage site at the N-terminal of PAR-2. Of the antibodies identified in this screen, antibody Ab3777 was found to have the highest affinity for PAR-2 (65 nM) in initial selection. Upon affinity maturation antibodies with up to 1000-fold improved affinities and dissociation constants (Kd) down to 50 μM were identified. The sequences of the heavy (IgG4) and light (lambda) chain variable regions of the antibody Ab3777 are presented below (CDR residues are underlined):

Ab3777 Lambda 3 Light Chain

(SEQ ID NO: 6)

DIELTQPPSV SVAPGQTARI SCSGDNLGKK YVQWYQQKPG
QAPVLVIYDD SNRPSGIPER FSGSNSGNTA TLTISGTQAE
DEADYYCSSW`DVGSDGWVFG GGTKLTVLGQ
Ab3777 VH3 Heavy Chain

(SEQ ID NO: 7)

QVQLVESGGG LVQPGGSLRL SCAASGFTFS`SYAMNWVRQA
PGKGLEWVST ISYSSSATSY`ADSVKGRFTI SRDNSKNTLY
LQMNSLRAED TAVYYCARIQ NDPMDVWGQG TLVTVSS
Ab4213 Lambda 3 Light Chain

(SEQ ID NO: 8)

DIELTQPPSV SVAPGQTARI SCSGDNIGTK YVYWYQQKPG
QAPVLVIYDD NNRPSGIPER FSGSNSGNTA TLTISGTQAE
DEADYYCQSY DSQTMVFGGG TKLTVLGQ
Ab4213 VH3 Heavy Chain

(SEQ ID NO: 9)

QVQLVESGGG LVQPGGSLRL SCAASGFTFS NHAMHWVRQA
PGKGLEWVSG ISGGGSNTYY ADSVKGRFTI SRDNSKNTLY
LQMNSLRAED TAVYYCARHG DGMDYFDFWG QGTLVTVSS

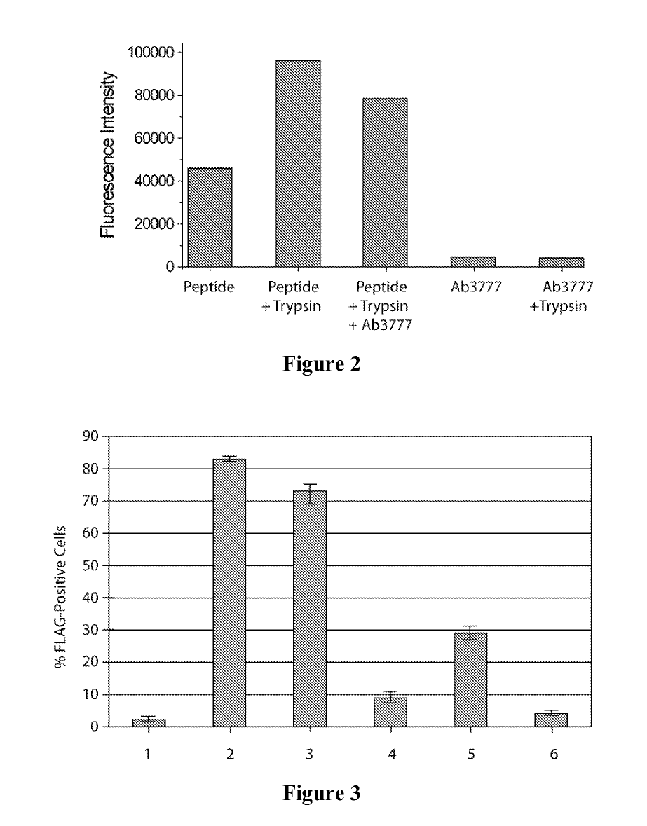

To assess the ability of antibody Ab3777 to block trypsin mediated cleavage of the PAR-2 N-terminus, antibodies were tested in a FRET assay that measures an increase in fluorescence upon cleavage of a labeled peptide representing the N-terminus of PAR-2. The results indicate that Ab3777 was able to bind to this peptide and partially block this cleavage in a transient manner (FIG. 2). Affinity matured antibodies completely inhibited peptide cleavage with IC₅₀s down to 30 nM which is most likely the sensitivity limit of the assay. To assess the ability of antibodies to block trypsin mediated cleavage of the PAR-2 N-terminus on cells, HEK transfectants over-expressing FLAG-tagged PAR-2 were treated with trypsin in the presence or absence of Ab3777 dHLX bivalent Fabs. Activation of PAR-2 results in cleavage/internalization of the receptor resulting in a loss of the FLAG epitope as detected by an antibody in a flow cytometry assay (FIG. 3). Table 1 provides the legend for FIG. 3.

TABLE 1

| FIG. 3 Legend: | | | | | |
|---|---|---|---|---|---|
| 1 | 2 | 3 | 4 | 5 | 6 |

Antibody Pretreatment:

| | | | | | | |
|---|---|---|---|---|---|---|
| Ab3777 DHLX | − | | + | − | + | − |
| Ab3207 (NC) DHLX | − | − | − | + | − | − |
| Trypsin Activation (30 nM; 5 minutes) | − | − | − | + | + | + |

Detection:

| | | | | | | |
|---|---|---|---|---|---|---|
| Anti-FLAG mAb_biotin | − | + | + | + | + | + |
| Streptavidin-PE | + | + | + | + | + | + |

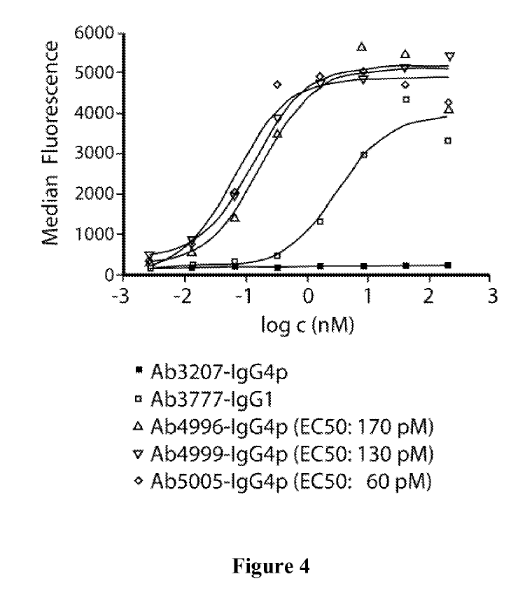

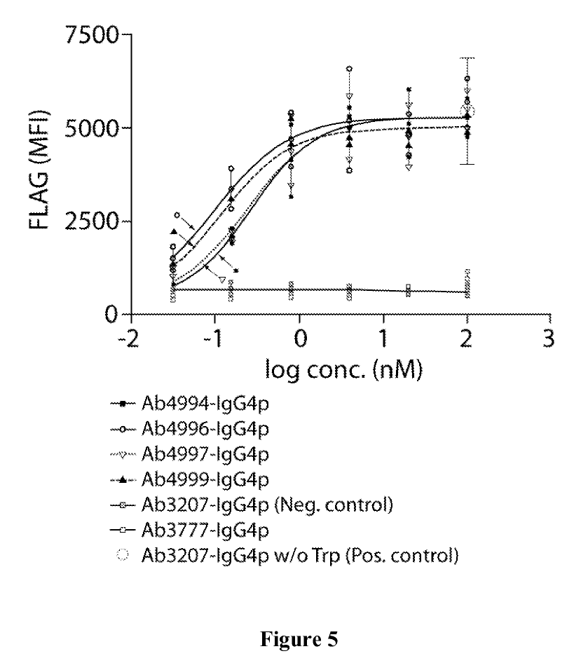

FIG. 4 shows the results of a study of the binding of affinity matured antibodies IgG4_Pro Ab4996, Ab4999, Ab5005 and the parental IgG1 Ab3777 to PAR-2 transfected HEK293 cells. FIG. 5 shows representative data of a FLAG-PAR-2 cleavage assay testing affinity-optimized IgGs. Detection of the FLAG-tag on the cell surface correlates with inhibitory activity.

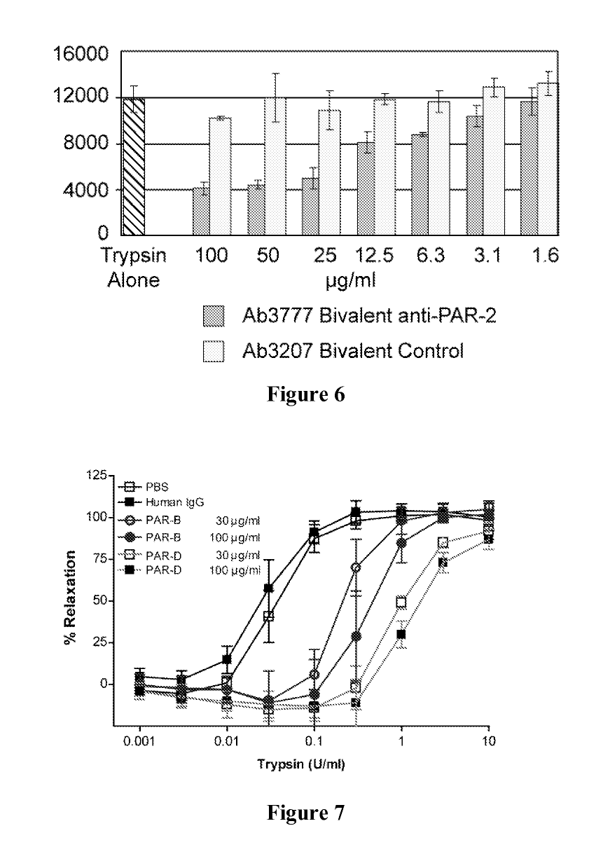

Ab3777 in the bivalent Fab format shows partial inhibition in a trypsin induced calcium response assay. Using A549 cells, PAR-2 activation was measured by detecting the calcium response following trypsin stimulation. Fabs were tested in this assay for their ability to inhibit this response. Ab3777, the Fab with the highest affinity for the PAR-2 P1 peptide, showed partial dose-dependent inhibition of the FLIPR when measured in the bivalent Fab format. This Fab was not active in the monovalent format and appears to be active but weaker in an IgG1 format. Control bivalent Fab (Ab3207) or IgG (LY6.3) do not bind PAR-2 and are included as negative controls. Antibody concentrations are expressed as μg/ml (FIG. 6).

### Example 2

### Affinity Maturation of Antibody Ab3777

Antibody Ab3777 was found to be uniquely capable of demonstrating partial inhibition in both a mechanistic (FRET-based PAR-2 peptide cleavage assay) and a functional assay (cell-based PAR-2 cleavage assay; cell-based trypsin stimulated calcium response assay). In order to increase the exhibited degree of inhibition so that it would inhibit cytokine production as measured in a longer term assay (e.g., trypsin induced IL-8 production by keratinocytes), derivatives of antibody Ab3777 exhibiting increased affinity to the PAR-2 N-terminal epitope were sought. Such antibodies would provide greater inhibition of PAR-2 cleavage and ultimately greater therapeutic benefit. Accordingly, affinity maturation of Ab3777 was undertaken. Maturation of a pool of other PAR-2 binding antibodies (including antibody Ab4213) exhibiting lower but significant affinity was also done, in parallel, to maximize the possibility of finding additional high affinity binders.

Characterization of 63 affinity matured Fabs led to the selection of eight candidates based on affinity, potency and maintenance of diversity, i.e. maturation from the parent or the pool as well as those with changes in the heavy chain or the light chain (Table 2).

TABLE 2

| Antibody | CDR | SEQ ID NO: | CDR Sequence |
|---|---|---|---|
| Ab3777 (parental) | H-CDR2 | 10 | STISYSSSAT SYADS |
| Ab4994 | H-CDR2 | 11 | SAISTSGHLT GYADS |
| Ab4997 | H-CDR2 | 12 | SAISYNGKLK GYADS |
| Ab5003 | H-CDR2 | 13 | SAISYKGHLT GYADS |
| Ab5006 | H-CDR2 | 14 | GAISFDGMLT GYADS |
| Ab5007 | H-CDR2 | 15 | SAISFSGHLT GYADS |
| Ab4213 (parental) | H-CDR2 | 16 | SGISGGGSNT YYADS |
| Ab4996 | H-CDR2 | 17 | SAISHPGKFT YYADS |
| Ab3777 (parental) | L-CDR3 | 18 | SSWDVGSDGW |
| Ab4999 | L-CDR3 | 19 | QTWDYSSIRD ETN |
| Ab5005 | L-CDR3 | 20 | QSWALVGSSE SN |

The matured binders Ab4994 and Ab4996 are derived from pannings on PAR-2 peptide in solution. All other matured binders (Ab4997, Ab4999, Ab5003, Ab5005, Ab5006, Ab5007) are derived from pannings on PAR-2 transfected cells.

The sequences of the heavy (IgG4) and light (lambda) chain variable regions of parental antibody Ab4213 and of matured antibodies Ab4994, Ab4996, Ab4997, Ab4999, Ab5003, Ab5005, Ab5006, and Ab5007 are presented below (CDR residues are underlined):

Ab4994 VH3 heavy chain

(SEQ ID NO: 21)

QVQLVESGGG LVQPGGSLRL SCAASGFTFS SYAMNWVRQA
PGKGLEWVSA ISYSGHLTGY ADSVKGRFTI SRDNSKNTLY
LQMNSLRAED TAVYYCARIQ NDPMDVWGQG TLVTVSS
Ab4996 VH3 heavy chain

(SEQ ID NO: 22)

QVQLVESGGG LVQPGGSLRL SCAASGFTFS NHAMHWVRQA
PGKGLEWVSA ISHPGKFTYY ADSVKGRFTI SRDNSKNTLY
LQMNSLRAED TAVYYCARHG DGMDYFDFWG QGTLVTVSS
Ab4997 VH3 heavy chain

(SEQ ID NO: 23)

QVQLVESGGG LVQPGGSLRL SCAASGFTFS SYAMNWVRQA
PGKGLEWVSA ISYNGKLKGY ADSVKGRFTI SRDNSKNTLY
LQMNSLRAED TAVYYCARIQ NDPMDVWGQG TLVTVSS
Ab5003 VH3 heavy chain

(SEQ ID NO: 24)

QVQLVESGGG LVQPGGSLRL SCAASGFTFS SYAMNWVRQA
PGKGLEWVSA ISYKGHLTGY ADSVKGRFTI SRDNSKNTLY
LQMNSLRAED TAVYYCARIQ NDPMDVWGQG TLVTVSS
Ab5006 VH3 heavy chain

(SEQ ID NO: 25)

QVQLVESGGG LVQPGGSLRL SCAASGFTFS SYAMNWVRQA
PGKGLEWVGA ISFDGMLTGY ADSVKGRFTI SRDNSKNTLY
LQMNSLRAED TAVYYCARIQ NDPMDVWGQG TLVTVSS
Ab5007 VH3 heavy chain

(SEQ ID NO: 26)

QVQLVESGGG LVQPGGSLRL SCAASGFTFS SYAMNWVRQA
PGKGLEWVSA ISFSGHLTGY ADSVKGRFTI SRDNSKNTLY
LQMNSLRAED TAVYYCARIQ NDPMDVWGQG TLVTVSS
Ab4999 lambda3 light chain

(SEQ ID NO: 27)

DIELTQPPSV SVAPGQTARI SCSGDNLGKK YVQWYQQKPG
QAPVLVIYDD SNRPSGIPER FSGSNSGNTA TLTISGTQAE
DEADYYCQTW DYSSIRDETN VFGGGTKLTV LGQ
Ab5005 lambda3 light chain

(SEQ ID NO: 28)

DIELTQPPSV SVAPGQTARI SCSGDNLGKK YVQWYQQKPG
QAPVLVIYDD SNRPSGIPER FSGSNSGNTA TLTISGTQAE
DEADYYCQSW ALVGSSESNV FGGGTKLTVL GQ

The sequence of the heavy chain of antibodies Ab4999 and Ab5005 is the same as that of the heavy chain of Ab3777 VH3 (SEQ ID NO: 7). The sequence of the light chain of antibodies Ab4994, Ab4997, Ab5003, Ab5006 and Ab5007 is the same as that of the light chain of Ab3777 lambda3 (SEQ ID NO: 6). The sequence of the light chain of antibody Ab4996 is the same as that of the light chain of Ab4213 lambda3 (SEQ ID NO: 8). Several cross-cloned (transfected) antibodies were also made: Ab5149 (composed of the light chain of Ab4999 combined with the heavy chain of Ab4997); Ab5150 (composed of the light chain of Ab4999 combined with the heavy chain of Ab5003) and Ab5151 (composed of the light chain of Ab4999 combined with the heavy chain of Ab5007).

Fab fragments of the above antibodies were also made. In each instance, the glutamine residue appearing at position 3 of the heavy chain variable sequences reported above was replaced with a glutamate residue:

Ab3777 VH3 Heavy Chain for Fab

(SEQ ID NO: 29)

QVELVESGGG LVQPGGSLRL SCAASGFTFS`SYAMNWVRQA
PGKGLEWVST ISYSSSATSY`ADSVKGRFTI SRDNSKNTLY
LQMNSLRAED TAVYYCARIQ NDPMDVWGQG TLVTVSS
Ab4213 VH3 Heavy Chain for Fab

(SEQ ID NO: 30)

QVELVESGGG LVQPGGSLRL SCAASGFTFS NHAMHWVRQA
PGKGLEWVSG ISGGGSNTYY ADSVKGRFTI SRDNSKNTLY
LQMNSLRAED TAVYYCARHG DGMDYFDFWG QGTLVTVSS
Ab4994 VH3 heavy chain for Fab

(SEQ ID NO: 31)

QVELVESGGG LVQPGGSLRL SCAASGFTFS SYAMNWVRQA
PGKGLEWVSA ISYSGHLTGY ADSVKGRFTI SRDNSKNTLY
LQMNSLRAED TAVYYCARIQ NDPMDVWGQG TLVTVSS
Ab4996 VH3 heavy chain for Fab

(SEQ ID NO: 32)

QVELVESGGG LVQPGGSLRL SCAASGFTFS NHAMHWVRQA
PGKGLEWVSA ISHPGKFTYY ADSVKGRFTI SRDNSKNTLY
LQMNSLRAED TAVYYCARHG DGMDYFDFWG QGTLVTVSS
Ab4997 VH3 heavy chain for Fab

(SEQ ID NO: 33)

QVELVESGGG LVQPGGSLRL SCAASGFTFS SYAMNWVRQA
PGKGLEWVSA ISYNGKLKGY ADSVKGRFTI SRDNSKNTLY
LQMNSLRAED TAVYYCARIQ NDPMDVWGQG TLVTVSS
Ab5003 VH3 heavy chain for Fab

(SEQ ID NO: 34)

QVELVESGGG LVQPGGSLRL SCAASGFTFS SYAMNWVRQA
PGKGLEWVSA ISYKGHLTGY ADSVKGRFTI SRDNSKNTLY
LQMNSLRAED TAVYYCARIQ NDPMDVWGQG TLVTVSS
Ab5006 VH3 heavy chain for Fab

(SEQ ID NO: 35)

QVELVESGGG LVQPGGSLRL SCAASGFTFS SYAMNWVRQA
PGKGLEWVGA ISFDGMLTGY ADSVKGRFTI SRDNSKNTLY
LQMNSLRAED TAVYYCARIQ NDPMDVWGQG TLVTVSS
Ab5007 VH3 heavy chain for Fab

(SEQ ID NO: 36)

QVELVESGGG LVQPGGSLRL SCAASGFTFS SYAMNWVRQA
PGKGLEWVSA ISFSGHLTGY ADSVKGRFTI SRDNSKNTLY
LQMNSLRAED TAVYYCARIQ NDPMDVWGQG TLVTVSS

The sequence of the heavy chain of Fab fragments of antibodies Ab4999 and Ab5005 is the same as that of the heavy chain of Ab3777 VH3 (SEQ ID NO: 29). The sequence of the light chain of Fab fragments of antibodies Ab4994, Ab4997, Ab5003, Ab5006 and Ab5007 is the same as that of the light chain of Ab3777 lambda3 (SEQ ID NO: 6). The sequence of the light chain of Fab fragments of antibody Ab4996 is the same as that of the light chain of Ab4213 lambda3 (SEQ ID NO: 8).

These Fabs were then converted to the IgG4_Pro (“IgG4p”) format. The IgG4_Pro antibodies consist of the variable VH and VL regions derived from corresponding Fabs fused to the constant regions of human IgG4 including a Ser241Pro mutation in the hinge region of the H-chain. The proline substitution in the hinge region is known to prevent the formation of half-molecules in natural human IgG4 molecules (Angal et al. (1993) Molec. Immunol. 30:105-108). These antibodies retained or showed improved binding (most likely due to avidity effects) to PAR-2 overexpressed on HEK cells (Table 3). Compared to the parental antibody Ab3777, the affinity matured antibodies displayed an up to approximately 1000 fold improvement in affinity. This demonstrates the effectiveness of affinity maturation for an antibody directed against a GPCR.

TABLE 3

| | | | Binding of | Binding of | |
|---|---|---|---|---|---|
| | | | Fab to | IgG4p to | |
| | | | HEK-PAR-2 | HEK- | Ratio |
| | | | (#3) | PAR-2 (#3) | of |
| | Parental | Optimized | (3RBO64) | (3RBO79) | EC₅₀ Fab |
| Antibody | Clone | CDR | EC₅₀ (nM) | EC₅₀ (nM) | EC₅₀ IgG |
| Ab4994 | Ab3777 | H-CDR2 | 1.90 | 0.40 | 4.8 |
| Ab4996 | Ab4213 | H-CDR2 | 0.10 | 0.17 | 0.6 |
| Ab4997 | Ab3777 | H-CDR2 | 3.03 | 0.38 | 8.0 |
| Ab4999 | Ab3777 | L-CDR3 | 0.06 | 0.20 | 0.3 |
| Ab5003 | Ab3777 | H-CDR2 | 7.03 | 0.46 | 15.3 |
| Ab5005 | Ab3777 | L-CDR3 | 0.17 | 0.06 | 2.8 |
| Ab5006 | Ab3777 | H-CDR2 | 11.90 | 0.17 | 70.0 |
| Ab5007 | Ab3777 | H-CDR2 | 9.66 | 0.50 | 19.3 |

Affinity matured and cross-cloned antibodies in the IgG4_Pro format were evaluated for cell binding, inhibition of trypsin induced calcium responses in a FLIPR assay, inhibition of PAR-2 peptide cleavage in a FRET assay and inhibition of PAR-2 cleavage in a cell based assay system. All candidates show excellent apparent affinity (EC50) for PAR-2 transfected cells and maximal efficacy and potency in all short term cellular assays (Table 4).

TABLE 4

| | | | | | FLAG Cell- |
|---|---|---|---|---|---|
| | | Cell Binding | | FRET | Based |
| | | HEK-PAR-2 | FLIPR | Peptide | Cleavage |
| | Parental | (#3) | A549 cells | Cleavage | Approx. IC₅₀ |
| Antibody | Clone | EC₅₀ (nM) | IC₅₀ (nM) | IC₅₀ (nM) | (nM) |
| Ab4994 | Ab3777 | 0.4 | 2.2 | <30 | <1 |
| Ab4996 | Ab4213 | 0.2 | 4.5 | <30 | <1 |
| Ab4997 | Ab3777 | 0.4 | 2.4 | <30 | <1 |
| Ab4999 | Ab3777 | 0.2 | 3.1 | <30 | <1 |
| Ab5003 | Ab3777 | 0.5 | 2.3 | <30 | <1 |
| Ab5005 | Ab3777 | 0.1 | 2.5 | <30 | <1 |
| Ab5007 | Ab3777 | 0.5 | 3.7 | <30 | <1 |
| Ab5149 | Ab3777 | 0.7 | 3.3 | <30 | <1 |
| Ab5150 | Ab3777 | 0.3 | 2.3 | <30 | <1 |
| Ab5151 | Ab3777 | 0.4 | 4.0 | <30 | <1 |

As shown in Table 4, this improvement in affinity translated to increased efficacy in assays that measured PAR-2 functional antagonism. Antibodies Ab4996, Ab4999, Ab5150 and Ab5151 were selected for further analysis.

To determine whether a further improvement in affinity or potency could be obtained, the optimized heavy and light chains from the 8 affinity matured antibodies were combined. Of the six additional antibodies generated, four showed good binding to PAR-2 transfected cells and inhibition in functional assays. Based on the diminished activity of Ab5006 and Ab5147 in the FLAG-based assay, these antibodies were excluded from further profiling. In sum, after the evaluation of 14 candidate antibodies for binding affinity to PAR-2 on cells, functional inhibition in FLIPR, blockade of cellular cleavage and blockade of peptide cleavage, ten candidates (seven selected from the initial affinity maturation and three from the cross-cloning) were identified. Four of the ten candidate antibodies (Ab4996, Ab4999, Ab5150 and Ab5151) were chosen for expression optimization. These antibodies were chosen on the basis of their excellent affinity and specificity for PAR-2, demonstration of robust inhibitory activity in short term cellular assays, maintenance of diversity (parental derivation, affinity matured vs. cross-cloned), and demonstration of species cross-reactivity.

### Example 3

### Optimization of Anti-PAR-2 Antibodies

Codon optimization of the variable region of the antibodies was employed to augment expression in the hamster cell line CHO-DG44, which was used for antibody production. The codon usage was adapted to the bias of hamster and RNA motifs which might interfere with RNA stability and expression were removed. The cDNA sequence of the optimized variable regions of three candidate anti-PAR-2 antibodies (Par-B, Par-C and Par-D) are presented below. Par-B and Par-C share the same light chain.

Antibody Par-B

SEQ ID NO:37 [optimized cDNA sequence of variable region of Ab4999 light chain].

gatatcgagc tgacccagcc ccccagcgtg agcgtggccc
caggccagac cgccaggatc agctgcagcg gcgacaacct
gggcaagaaa tacgtgcagt ggtatcagca gaagcccggc
caggcccccg tgctggtgat ctacgacgac agcaacaggc
ccagcggcat ccccgagagg ttcagcggca gcaacagcgg
caacaccgcc accctgacca tcagcggcac ccaggccgag
gacgaggccg actactactg ccagacctgg gactacagca
gcatcaggga cgagaccaac gtgttcggcg gagggaccaa
gttaaccgtc ctaggtcag

SEQ ID NO:38 [optimized cDNA sequence of variable region of Ab4999 heavy chain].

caggtggagc tggtcgagag cggcggaggg ctggtgcagc
caggcggcag cctgaggctg tcctgcgccg cctccggatt
caccttcagc agctacgcca tgaactgggt gcggcaggcc
ccaggcaagg gcctggagtg ggtgtccacc atcagctaca
gcagcagcgc cacctcctac gccgacagcg tgaagggcag
gttcaccatc agcagggaca acagcaagaa caccctgtac
ctgcagatga acagcctgag ggccgaggac accgccgtgt
actactgcgc caggatccag aacgacccca tggatgtgtg
gggccagggc accctggtga cagtgagctc a

Antibody Par-C

SEQ ID NO:37 [optimized cDNA sequence of variable region of Ab4999 light chain].
SEQ ID NO:39 [optimized cDNA sequence of variable region of Ab5007 or Ab5151 heavy chain].

caggtggagc tggtcgagag cggcggaggg ctggtgcagc
caggcggcag cctgaggctg tcctgcgccg cctccggatt
caccttcagc agctacgcca tgaactgggt gcggcaggcc
ccaggcaagg gcctggagtg ggtgtccgcc atcagcttca
gcggccacct gaccggctac gccgacagcg tgaagggcag
gttcaccatc agcagggaca acagcaagaa caccctgtac
ctgcagatga acagcctgag ggccgaggac accgccgtgt
actactgcgc caggatccag aacgacccca tggatgtgtg
gggccagggc accctggtga cagtgagctc a

Antibody Par-D

SEQ ID NO:40 [optimized cDNA sequence of variable region of Ab4996 light chain].

gatatcgagc tgacccagcc ccccagcgtg agcgtggccc
caggccagac cgccaggatc agctgcagcg gcgacaacat
cggcaccaag tacgtgtact ggtatcagca gaagcccggc
caggcccccg tgctggtgat ctacgacgac aacaacaggc
ccagcggcat ccccgagagg ttcagcggca gcaacagcgg
caacaccgcc accctgacca tcagcggcac ccaggccgag
gacgaggccg actactactg ccagagctac gacagccaga
ccatggtgtt cggcggaggg accaagttaa ccgtgctggg acag

SEQ ID NO:41 [optimized cDNA sequence of variable region of Ab4996 heavy chain].

caggtggagc tggtcgagag cggcggaggg ctggtgcagc
caggcggcag cctgaggctg tcctgcgccg cctccggatt
caccttcagc aaccacgcca tgcactgggt gcggcaggcc
ccaggcaagg gcctggagtg ggtgtccgcc atcagccacc
ccggcaagtt cacctactac gccgacagcg tgaagggcag
gttcaccatc agcagggaca acagcaagaa caccctgtac
ctgcagatga acagcctgag ggccgaggac accgccgtgt
actactgcgc caggcacggc gacggcatgg actacttcga
cttttggggc cagggcaccc tggtgacagt gagctca

### Example 4

### Binding of Optimized Anti-PAR-2 Antibodies To Primary Target: PAR-2 Peptide And Cell Binding

The antibody PAR-2 effector molecule candidates were evaluated for their ability to immunospecifically bind to an N-terminal PAR-2 peptide sequence that includes the trypsin cleavage site. In order to determine the binding affinity to this epitope, peptides derived from PAR-2 sequences of different species were employed (SEQ ID NOs: 1-4, above). Binding to peptide in solution was determined by Kinexa technology. As shown in Table 5, all four antibodies displayed high affinities to the human PAR-2 peptide with dissociation constants (Kd) in the range of 30 to 200 μM. Antibodies Par-B and Par-C revealed similar affinities to the mouse PAR-2 peptide, while antibody Par-D showed somewhat weaker binding and showed approximately 100-fold lower binding compared to the human peptide. In addition, affinities of antibodies Par-B and Par-D to peptides derived from rat and cynomolgus PAR-2 were measured and showed dissociation constants in a similar range as to the human PAR-2 peptide.

TABLE 5
Kd Values (nM) Determined for PAR-2 Antibodies

| | Par-B | Par-C | Par-D |
|---|---|---|---|
| PAR-2 Peptide | (n = 1) | (n = 1) | (n = 2) |
| SEQ ID NO: 1 (Human) | 0.08 | 0.03 | 0.15 |
| SEQ ID NO: 2 (Murine) | 0.08 | 0.03 | 0.42 |
| SEQ ID NO: 3 (Rat) | 0.02 | n.d. | 0.04 |
| SEQ ID NO: 4 (Cynomolgus) | 0.04 | n.d. | 0.14 |

n.d.—not done; (n = number of trials)

The unmodified antibodies Ab4996, Ab4999 and Ab5151 exhibited concentration-dependent binding to human PAR-2 transfected HEK293 cells with EC₅₀ values of 0.2 nM (antibodies Ab4996 and Ab4999) and 0.4 nM (antibody Ab5151). Consistent with these data, candidate antibodies Par-B and Par-D showed specific saturable binding to human PAR-2 transfected HEK293 cells. Antibodies Par-B and Par-D both displayed potent binding activity with EC₅₀ values of approximately 0.3 nM and approximately 0.4 nM (n=2), respectively. Binding with about 3 to 5-fold lower maximal intensity was also detected on the parental HEK293 cell line which is reported to express endogenous PAR-2. Additionally, specific binding of antibodies to the human lung epithelial cell lineA549 expressing endogenous PAR-2 was detected and could be inhibited by adding the PAR-2 peptide derived from the N-terminal region of PAR-2.

### Example 5

### Molecular Selectivity of Anti-PAR-2 Antibodies

The molecular selectivity of antibodies for PAR-2 versus PAR-1 and PAR-4 was assessed using peptides derived from the N-terminal regions of all three PARs:

| | |
|---|---|
| PAR-2: | |
| (SEQ ID NO: 42) | SKGRSLIGKVDGTSHVTGKGV |
| PAR-1: | |
| (SEQ ID NO: 43) | LDPRSFLLRNPNDKYEPFWEDEEKNESGL |
| PAR-4: | |
| (SEQ ID NO: 44) | PAPRGYPGQVSANDSDTLELPDSSRALL |
| | (Cys at position 11 has been |
| | replaced by Ser) |

ELISA experiments demonstrated specific binding of Ab4996, Ab4999 and Ab5151 to PAR-2 peptide (coupled to Transferrin as carrier), but no binding to PAR-4 peptide. Ab4996 showed limited binding (about 100-fold weaker) to the PAR-1 peptide, whereas Ab4999 and Ab5151 did not exhibit detectable binding to the PAR-1 peptide.

Thrombin activates PAR-1, whereas PAR-2 is activated by trypsin in A549 cells. Thus, to assess the functional relevance of the observed reactivity of Ab4996 with the PAR-1 peptide, the ability of Ab4996 to inhibit a thrombin-induced calcium response was analyzed using a FLIPR assay. Ab4996 and Ab5151 displayed significant inhibition of the trypsin mediated calcium response, but no inhibition of the thrombin induced response at antibody concentrations up to 300 nM. These studies indicate that the observed low level ability of Ab4996 to bind to a PAR-1 peptide is not physiologically relevant, and that the isolated antibodies are selective for PAR-2 and do not interfere with activation of PAR-1.

### Example 6

### Cellular Activity of PAR-2 Antibodies

Antibodies Par-B, Par-C and Par-D all showed potent inhibition of calcium release in the human lung epithelial cell line A549 stimulated with trypsin as demonstrated with their parental antibodies.

The cellular activity of antibodies Par-B and Par-D was assessed in a FLIPR assay with human primary keratinocytes. The cellular activity of antibodies Par-B and Par-D was assessed in a FLIPR assay with human primary keratinocytes. In a 384-well flat bottom tissue culture plate, human keratinocytes (2×10⁵ cells) were incubated overnight at 37° C. and 5% CO₂. Following the removal of culture media, the confluent cell layer was loaded with Fluo-3, AM cell permeant dye. After washing the cell layer 2×, human keratinocytes were incubated with varying concentrations of antibodies Par-B and Par-D for 30 minutes and then stimulated with 0.14 μM of trypsin. Cellular calcium flux was measured using a fluorescent imaging plate reader (FLIPR). Both antibodies dose-dependently inhibited trypsin mediated calcium flux in human primary keratinocytes with IC50 values in the low nanomolar range comparable to the activity seen with A549 cells. Both antibodies showed efficient concentration-dependent inhibition with IC₅₀ values in the low nanomolar range comparable to the activity seen with A549 cells.

To further assess functional cross-reactivity with mouse PAR-2, the antibodies were tested in FLIPR assays with the mouse Lewis lung carcinoma (LLC) cell line. Antibodies Par-B and Par-C showed efficient inhibition of trypsin-induced calcium release in LLC cells, whereas antibody Par-D did not exhibit a significant effect. The results of the FLIPR assays are shown in Table 6.

TABLE 6

| | IC₅₀ Values (nM) (SD) | | |
|---|---|---|---|
| Cell line: | Par-B | Par-C | Par-D |
| Human A549 | 0.6 (0.6) | 1.0 (0.8) | 1.8 (0.7) |
| Human keratinocytes | 1.1 (0.9) | n.d. | 2.9 (1.9) |
| Mouse LLC cells | 0.4 (0.1) | 0.7 (0.2) | no effect |

n.d.—not done; SD—standard deviation

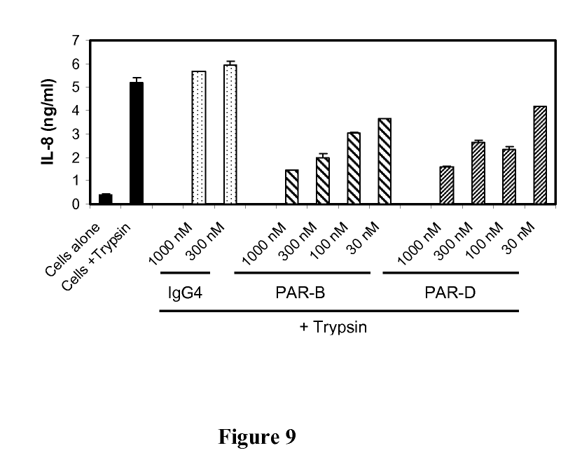

In a second, longer term cell-based assay, the secretion of IL-8 was analyzed in human primary keratinocytes that had been stimulated with trypsin for approximately 20 hours. In agreement with the results of the above-described FLIPR assay, a clear concentration-dependent inhibition of the IL-8 secretion was observed (FIG. 9). However, in contrast to the FLIPR assay, the potency of the antibody PAR-2 effector molecule candidates was approximately 100-fold lower.

The IC₅₀ in this assay was approximately 100 nM. However, this is only an estimation since a complete dose-response curve was not obtained. The lower potency in the IL-8 assay (relative to the FLIPR assay) may be explained by the longer incubation with trypsin, which competes with antibody for the same binding site on PAR-2.

In summary, the inhibitory activity of the preferred anti-PAR-2 antibodies was demonstrated in two different cellular assays (measuring calcium release or IL-8 secretion). The results of the latter assay suggest that human keratinocytes are suitable targets and antibody mediated inhibition of the neutrophil attracting chemokine IL-8 could provide therapeutic benefit in psoriasis. A summary of the assay results is reported in Table 7.

TABLE 7

| | Summary of Cellular Activity (IC₅₀ Values | |
|---|---|---|
| | in nM (SD)) of Anti-PAR-2 Antibody | |
| Assay System | Par-B | Par-D |
| FLIPR human cells * | 0.6 (0.6) | 1.8 (0.7) |
| FLIPR mouse cells ** | 0.4 (0.1) | no activity |
| FLIPR rat cells *** | 1.1 (0.7) | 1.0 (0.3) |
| Human Keratinocyte/ | Approx. ~100 nM | Approx. ~100 nM |
| IL-8 assay | | |

* human epithelial cell line A549
** mouse epithelial cell line LLC; relative to human
*** rat epithelial cell line ARIP; relative to human

### Example 7

### Species Cross-Reactivity and Aortic Ring Assays

The analysis of cross-reactivity with non-human species is desirable to facilitate the identification of relevant species that may be employed in pharmacological and toxicological studies. The above-described PAR-2 peptide binding and cellular FLIPR assays were used to assess the cross-reactivity of the anti-human PAR-2 antibodies with rodent PAR-2.

As described above, both antibodies Par-B and Par-D showed sub-nanomolar affinities to the murine PAR-2 peptide that were comparable to the observed affinities to the human peptide, although antibody Par-D appeared to have a slightly lower binding activity on the mouse peptide. FLIPR studies with the murine LLC cell line described above demonstrated potent inhibitory activity of antibody Par-B, but no effect of antibody Par-D.

In order to assess the functional cross-reactivity of these antibodies against non-human PAR-2, an aortic ring assay was performed with primary rodent tissue (see: Saifeddine M. et al. (1996) Br. J. Pharmacol 118:521-530; Kamata, K. et al. (1997) Res. Commun Mol. Pathol. Pharmacol. 96:319-328). This assay measures the inhibitory activity of PAR-2 antibodies on the trypsin-induced relaxation of rat or mouse aortic ring tissue. Trypsin as well as PAR-2 agonistic peptides were found to be able to relax pre-constricted aortic rings presumably through the PAR-2 mediated generation of nitrous oxide (NO) and endothelium derived hyperpolarizing factor (EDHF) (FIG. 7). Antibodies Par-B and Par-D were each found to display potent inhibition of trypsin-induced relaxation of rat aortic rings, whereas the relaxation of mouse aortic rings was only inhibited by antibody Par-B and not by antibody Par-D. These data are consistent with the above-described data from FLIPR studies using mouse and rat cell lines. In conclusion, antibody Par-B displays functional cross-reactivity with both mouse and rat PAR-2, while antibody Par-D shows functional activity only with rat, but not with mouse, PAR-2. Species cross-reactivity studies are summarized in Table 8 below.

TABLE 8

| Assay | Par-B | Par-D |
|---|---|---|
| Mouse PAR-2 Peptide | 1-fold vs. human peptide | 0.4-fold vs. human peptide |
| Binding | (Kd = 0.08 nM) | (Kd = 0.42 nM) |
| Mouse Cell Line FLIPR | IC50 Approximately 0.4 nM | No inhibition at concentrations |
| | | up to 200 nM |
| Mouse Aortic Ring Assay | Approximately 5 fold shift | No inhibition at concentrations |
| | EC50 (Trypsin) at Ab conc. | of 200 and 670 nM |
| | 200 nM | |
| Rat PAR-2 Peptide Binding | 4-fold vs. human peptide | 4-fold vs. human peptide |
| | (Kd = 0.02 nM) | (Kd = 0.04 nM) |
| Rat Aortic Ring Assay | Approximately 8 fold shift | Approximately 30 fold shift |
| | EC50 (Trypsin) at Ab conc. | EC50 (Trypsin) at Ab conc. |
| | 200 nM | 200 nM |

### Example 8

### Activity of PAR-2 Antibodies in the Trans Vivo DTH Model

The trans vivo delayed type hypersensitivity (DTH) is a validated model of human T cell mediated inflammation in mice (Warnecke, G. et al. (2007) Transplantation 84(3):392-399; Pelletier, R. P. et al. (Mar. 28, 2007, Epub) Hum. Immunol. 68(6):514-522; Carrodeguas, L. et al. (1999) Hum. Immunol. 60(8):640-651). In this assay, human PBMCs from tetanus-sensitized donors are isolated and co-injected with tetanus toxoid (TT) into the mouse footpad. The swelling response that develops in the footpad is measured at 24 hours. This swelling response is dependent on antigen-specific activation of the human T cells as well as on the inflammation mediated by mouse cells.

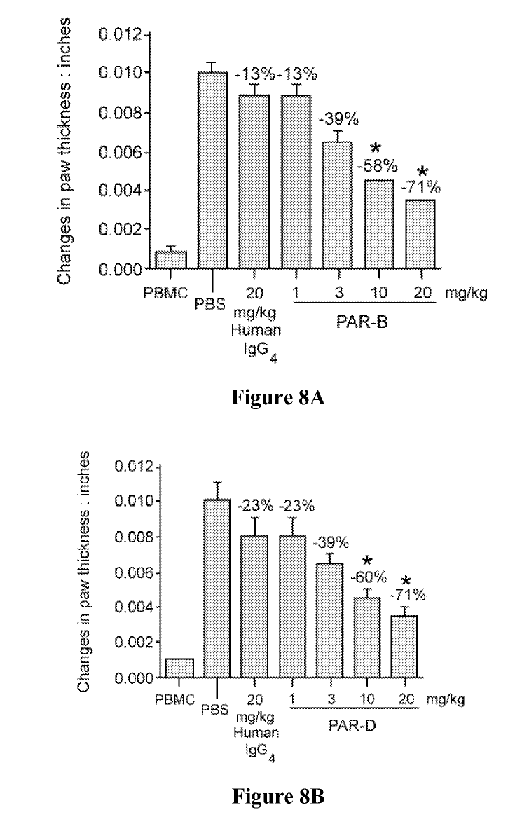

Shown in FIG. 8A and FIG. 8B, are the changes in footpad thickness 24 hours after injection of PBMCs and TT. Antibodies Par-B (FIG. 8A) and Par-D (FIG. 8B) inhibited the response dose-dependently (71% inhibition at 20 mg/kg) with an efficacy similar to that of dexamethasone at 10 mg/kg (approximately 80% inhibition). These experiments demonstrate the ability of anti-PAR-2 antibodies to functionally inhibit in vivo a cell mediated immune response.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A Protease Receptor-2 (PAR-2) effector molecule comprising a domain capable of binding to a region of PAR-2, wherein said binding modulates the activation or activity of PAR-2.

2. The PAR-2 effector molecule of claim 1, wherein said molecule is a negative effector of PAR-2 activation or activity.

3. The PAR-2 effector molecule of claim 2, wherein said molecule is an antibody.

4. The PAR-2 effector molecule of claim 3, wherein said antibody is an engineered antibody.

5. The PAR-2 effector molecule of claim 4, wherein said engineered antibody exhibits a K_{d} of from about 0.03 nM to about 0.2 nM.

6. The PAR-2 effector molecule of claim 4, wherein said engineered antibody exhibits concentration-dependent binding to human PAR-2 transfected HEK293 cells with an EC₅₀ of from about 0.2 nM to about 0.4 nM.

7. The PAR-2 effector molecule of claim 4, wherein said engineered antibody inhibits trypsin-induced calcium release, in a cell of a human, mouse or rat epithelial cell line, with an IC₅₀ of from about 2.0 nM to about 0.5 nM.

8. The PAR-2 effector molecule of claim 7, wherein said IC₅₀ is from about 1.0 nM to about 0.5 nM.

9. The PAR-2 effector molecule of claim 4, wherein said engineered antibody exhibits at least 100-fold greater binding affinity to human PAR-2 relative to human PAR-1.

10. The PAR-2 effector molecule of claim 4, wherein said engineered antibody comprises a heavy chain CDR having the sequence of any of SEQ ID NOs: 11-15 or 17.

11. The PAR-2 effector molecule of claim 4, wherein said engineered antibody comprises a light chain CDR having the sequence of any of SEQ ID NOs: 19-20.

12. The PAR-2 effector molecule of claim 4, wherein said engineered antibody comprises a heavy chain having the sequence of any of SEQ ID NOs: 21-26 or SEQ ID NOs: 30-36.

13. The PAR-2 effector molecule of claim 4, wherein said engineered antibody comprises a light chain having the sequence of any of SEQ ID NOs: 27-28.

14. The PAR-2 effector molecule of claim 4, wherein said engineered antibody is Par-B, Par-C or Par-D.

15. The PAR-2 effector molecule of claim 4, having an antibody light chain whose amino acid sequence is encoded by an expressed DNA sequence selected from the DNA sequences of SEQ ID NO: 37 or SEQ ID NO: 40.

16. The PAR-2 effector molecule of claim 4, having a heavy chain whose amino acid sequence is encoded by an expressed DNA sequence selected from the DNA sequences of SEQ ID NO: 38, SEQ ID NO: 39 or SEQ ID NO: 41.

17. A pharmaceutical composition comprising a PAR-2 effector molecule of claim 1 and a pharmacologically acceptable excipient.

18. The pharmaceutical composition of claim 17, wherein said composition additionally comprises an additional anti-inflammatory agent.

19. A method for treating inflammation in a recipient mammal, comprising administering to said mammal a PAR-2 effector molecule of claim 1 and a pharmacologically acceptable excipient, in an amount sufficient to provide such treatment.

20. The method of claim 19, wherein said composition additionally comprises an additional anti-inflammatory agent.

21. The method of claim 19, wherein said inflammation is selected from the group consisting of: psoriasis, contact dermatitis, inflammatory bowel disease, trans vivo delayed type hypersensitivity, PAR-2 mediated aortic ring relaxation and pain.

22. A method for preventing or inhibiting inflammation in a recipient mammal, comprising administering to said mammal, in advance of said inflammation, a pharmaceutical composition, comprising the PAR-2 effector molecule of claim 1 and a pharmacologically acceptable excipient, in an amount sufficient to provide such prevention or inhibition.

23. The method of claim 22, wherein said composition additionally comprises an additional anti-inflammatory agent.

24. The method of claim 22, wherein said inflammation is selected from the group consisting of: psoriasis, contact dermatitis, inflammatory bowel disease, trans vivo delayed type hypersensitivity, PAR-2 mediated aortic ring relaxation and pain.


SEQUENCE LISTING (44 sequences)

SEQ ID NO: 1 - protein, 397 aa, Homo sapiens
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 2 - protein, 21 aa, Mus musculus
SKGRSLIGRLETQPPITGKGV

SEQ ID NO: 3 - protein, 21 aa, Rattus norvegicus
SKGRSLIGRLDTPPPITGKGA

SEQ ID NO: 4 - protein, 21 aa, Macaca fascicularis
SKGRSLIGRVDGTFHVTGKGV

SEQ ID NO: 5 - protein, 21 aa, Artificial
  Consensus sequence of PAR-2 fragment
SKGRSLIGRLDTTPPITGKGV

SEQ ID NO: 6 - protein, 110 aa, Artificial
  Antibody identified via phage display
DIELTQPPSVSVAPGQTARISCSGDNLGKKYVQWYQQKPGQAPVLVIYDDSNRPSGIPERFSGSNSGNTATLTISGTQAEDEADYYCSSWDVGSDGWVFGGGTKLTVLGQ

SEQ ID NO: 7 - protein, 117 aa, Artificial
  Antibody identified via phage display
QVQLVESGGGLVQPGGSLRLSCAASGFTFSSYAMNWVRQAPGKGLEWVSTISYSSSATSYADSVKGRFTISRDNSKNTLYLQMNSLRAEDTAVYYCARIQNDPMDVWGQGTLVTVSS

SEQ ID NO: 8 - protein, 108 aa, Artificial
  Antibody identified via phage display
DIELTQPPSVSVAPGQTARISCSGDNIGTKYVYWYQQKPGQAPVLVIYDDNNRPSGIPERFSGSNSGNTATLTISGTQAEDEADYYCQSYDSQTMVFGGGTKLTVLGQ

SEQ ID NO: 9 - protein, 119 aa, Artificial
  Antibody identified via phage display
QVQLVESGGGLVQPGGSLRLSCAASGFTFSNHAMHWVRQAPGKGLEWVSGISGGGSNTYYADSVKGRFTISRDNSKNTLYLQMNSLRAEDTAVYYCARHGDGMDYFDFWGQGTLVTVSS

SEQ ID NO: 10 - protein, 15 aa, Artificial
  Antibody identified via phage display
STISYSSSATSYADS

SEQ ID NO: 11 - protein, 15 aa, Artificial
  Antibody modified via affinity maturation
SAISTSGHLTGYADS

SEQ ID NO: 12 - protein, 15 aa, Artificial
  Antibody modified via affinity maturation
SAISYNGKLKGYADS

SEQ ID NO: 13 - protein, 15 aa, Artificial
  Antibody modified via affinity maturation
SAISYKGHLTGYADS

SEQ ID NO: 14 - protein, 15 aa, Artificial
  Antibody modified via affinity maturation
GAISFDGMLTGYADS

SEQ ID NO: 15 - protein, 15 aa, Artificial
  Antibody modified via affinity maturation
SAISFSGHLTGYADS

SEQ ID NO: 16 - protein, 15 aa, Artificial
  Antibody identified via phage display
SGISGGGSNTYYADS

SEQ ID NO: 17 - protein, 15 aa, Artificial
  Antibody modified via affinity maturation
SAISHPGKFTYYADS

SEQ ID NO: 18 - protein, 10 aa, Artificial
  Antibody identified via phage display
SSWDVGSDGW

SEQ ID NO: 19 - protein, 13 aa, Artificial
  Antibody modified via affinity maturation
QTWDYSSIRDETN

SEQ ID NO: 20 - protein, 12 aa, Artificial
  Antibody modified via affinity maturation
QSWALVGSSESN

SEQ ID NO: 21 - protein, 117 aa, Artificial
  Antibody modified via affinity maturation
QVQLVESGGGLVQPGGSLRLSCAASGFTFSSYAMNWVRQAPGKGLEWVSAISYSGHLTGYADSVKGRFTISRDNSKNTLYLQMNSLRAEDTAVYYCARIQNDPMDVWGQGTLVTVSS

SEQ ID NO: 22 - protein, 119 aa, Artificial
  Antibody modified via affinity maturation
QVQLVESGGGLVQPGGSLRLSCAASGFTFSNHAMHWVRQAPGKGLEWVSAISHPGKFTYYADSVKGRFTISRDNSKNTLYLQMNSLRAEDTAVYYCARHGDGMDYFDFWGQGTLVTVSS

SEQ ID NO: 23 - protein, 117 aa, Artificial
  Antibody modified via affinity maturation
QVQLVESGGGLVQPGGSLRLSCAASGFTFSSYAMNWVRQAPGKGLEWVSAISYNGKLKGYADSVKGRFTISRDNSKNTLYLQMNSLRAEDTAVYYCARIQNDPMDVWGQGTLVTVSS

SEQ ID NO: 24 - protein, 117 aa, Artificial
  Antibody modified via affinity maturation
QVQLVESGGGLVQPGGSLRLSCAASGFTFSSYAMNWVRQAPGKGLEWVSAISYKGHLTGYADSVKGRFTISRDNSKNTLYLQMNSLRAEDTAVYYCARIQNDPMDVWGQGTLVTVSS

SEQ ID NO: 25 - protein, 117 aa, Artificial
  Antibody modified via affinity maturation
QVQLVESGGGLVQPGGSLRLSCAASGFTFSSYAMNWVRQAPGKGLEWVGAISFDGMLTGYADSVKGRFTISRDNSKNTLYLQMNSLRAEDTAVYYCARIQNDPMDVWGQGTLVTVSS

SEQ ID NO: 26 - protein, 117 aa, Artificial
  Antibody modified via affinity maturation
QVQLVESGGGLVQPGGSLRLSCAASGFTFSSYAMNWVRQAPGKGLEWVSAISFSGHLTGYADSVKGRFTISRDNSKNTLYLQMNSLRAEDTAVYYCARIQNDPMDVWGQGTLVTVSS

SEQ ID NO: 27 - protein, 113 aa, Artificial
  Antibody modified via affinity maturation
DIELTQPPSVSVAPGQTARISCSGDNLGKKYVQWYQQKPGQAPVLVIYDDSNRPSGIPERFSGSNSGNTATLTISGTQAEDEADYYCQTWDYSSIRDETNVFGGGTKLTVLGQ

SEQ ID NO: 28 - protein, 112 aa, Artificial
  Antibody modified via affinity maturation
DIELTQPPSVSVAPGQTARISCSGDNLGKKYVQWYQQKPGQAPVLVIYDDSNRPSGIPERFSGSNSGNTATLTISGTQAEDEADYYCQSWALVGSSESNVFGGGTKLTVLGQ

SEQ ID NO: 29 - protein, 117 aa, Artificial
  Antibody identified via phage display
QVELVESGGGLVQPGGSLRLSCAASGFTFSSYAMNWVRQAPGKGLEWVSTISYSSSATSYADSVKGRFTISRDNSKNTLYLQMNSLRAEDTAVYYCARIQNDPMDVWGQGTLVTVSS

SEQ ID NO: 30 - protein, 119 aa, Artificial
  Antibody identified via phage display
QVELVESGGGLVQPGGSLRLSCAASGFTFSNHAMHWVRQAPGKGLEWVSGISGGGSNTYYADSVKGRFTISRDNSKNTLYLQMNSLRAEDTAVYYCARHGDGMDYFDFWGQGTLVTVSS

SEQ ID NO: 31 - protein, 117 aa, Artificial
  Antibody modified via affinity maturation
QVELVESGGGLVQPGGSLRLSCAASGFTFSSYAMNWVRQAPGKGLEWVSAISYSGHLTGYADSVKGRFTISRDNSKNTLYLQMNSLRAEDTAVYYCARIQNDPMDVWGQGTLVTVSS

SEQ ID NO: 32 - protein, 119 aa, Artificial
  Antibody modified via affinity maturation
QVELVESGGGLVQPGGSLRLSCAASGFTFSNHAMHWVRQAPGKGLEWVSAISHPGKFTYYADSVKGRFTISRDNSKNTLYLQMNSLRAEDTAVYYCARHGDGMDYFDFWGQGTLVTVSS

SEQ ID NO: 33 - protein, 117 aa, Artificial
  Antibody modified via affinity maturation
QVELVESGGGLVQPGGSLRLSCAASGFTFSSYAMNWVRQAPGKGLEWVSAISYNGKLKGYADSVKGRFTISRDNSKNTLYLQMNSLRAEDTAVYYCARIQNDPMDVWGQGTLVTVSS

SEQ ID NO: 34 - protein, 117 aa, Artificial
  Antibody modified via affinity maturation
QVELVESGGGLVQPGGSLRLSCAASGFTFSSYAMNWVRQAPGKGLEWVSAISYKGHLTGYADSVKGRFTISRDNSKNTLYLQMNSLRAEDTAVYYCARIQNDPMDVWGQGTLVTVSS

SEQ ID NO: 35 - protein, 117 aa, Artificial
  Antibody modified via affinity maturation
QVELVESGGGLVQPGGSLRLSCAASGFTFSSYAMNWVRQAPGKGLEWVGAISFDGMLTGYADSVKGRFTISRDNSKNTLYLQMNSLRAEDTAVYYCARIQNDPMDVWGQGTLVTVSS

SEQ ID NO: 36 - protein, 117 aa, Artificial
  Antibody modified via affinity maturation
QVELVESGGGLVQPGGSLRLSCAASGFTFSSYAMNWVRQAPGKGLEWVSAISFSGHLTGYADSVKGRFTISRDNSKNTLYLQMNSLRAEDTAVYYCARIQNDPMDVWGQGTLVTVSS

SEQ ID NO: 37 - DNA, 339 bp, Artificial
  Optimized cDNA sequence of variable region of Ab4999 light chain
gatatcgagctgacccagccccccagcgtgagcgtggccccaggccagaccgccaggatcagctgcagcggcgacaacctgggcaagaaatacgtgcagtggtatcagcagaagcccggccaggcccccgtgctggtgatctacgacgacagcaacaggcccagcggcatccccgagaggttcagcggcagcaacagcggcaacaccgccaccctgaccatcagcggcacccaggccgaggacgaggccgactactactgccagacctgggactacagcagcatcagggacgagaccaacgtgttcggcggagggaccaagttaaccgtcctaggtcag

SEQ ID NO: 38 - DNA, 351 bp, Artificial
  Optimized cDNA sequence of variable region of Ab4999 heavy chain
caggtggagctggtcgagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcagcagcgccacctcctacgccgacagcgtgaagggcaggttcaccatcagcagggacaacagcaagaacaccctgtacctgcagatgaacagcctgagggccgaggacaccgccgtgtactactgcgccaggatccagaacgaccccatggatgtgtggggccagggcaccctggtgacagtgagctca

SEQ ID NO: 39 - DNA, 351 bp, Artificial
  Optimized cDNA sequence of variable region of Ab5007 or Ab5151 heavy chain
caggtggagctggtcgagagcggcggagggctggtgcagccaggcggcagcctgaggctgtcctgcgccgcctccggattcaccttcagcagctacgccatgaactgggtgcggcaggccccaggcaagggcctggagtgggtgtccgccatcagcttcagcggccacctgaccggctacgccgacagcgtgaagggcaggttcaccatcagcagggacaacagcaagaacaccctgtacctgcagatgaacagcctgagggccgaggacaccgccgtgtactactgcgccaggatccagaacgaccccatggatgtgtggggccagggcaccctggtgacagtgagctca

SEQ ID NO: 40 - DNA, 324 bp, Artificial
  Optimized cDNA sequence of variable region of Ab4996 light chain
gatatcgagctgacccagccccccagcgtgagcgtggccccaggccagaccgccaggatcagctgcagcggcgacaacatcggcaccaagtacgtgtactggtatcagcagaagcccggccaggcccccgtgctggtgatctacgacgacaacaacaggcccagcggcatccccgagaggttcagcggcagcaacagcggcaacaccgccaccctgaccatcagcggcacccaggccgaggacgaggccgactactactgccagagctacgacagccagaccatggtgttcggcggagggaccaagttaaccgtgctgggacag

SEQ ID NO: 41 - DNA, 357 bp, Artificial
  Optimized cDNA sequence of variable region of Ab4996 heavy chain
caggtggagctggtcgagagcggcggagggctggtgcagccaggcggcagcctgaggctgtcctgcgccgcctccggattcaccttcagcaaccacgccatgcactgggtgcggcaggccccaggcaagggcctggagtgggtgtccgccatcagccaccccggcaagttcacctactacgccgacagcgtgaagggcaggttcaccatcagcagggacaacagcaagaacaccctgtacctgcagatgaacagcctgagggccgaggacaccgccgtgtactactgcgccaggcacggcgacggcatggactacttcgacttttggggccagggcaccctggtgacagtgagctca

SEQ ID NO: 42 - protein, 21 aa, Homo sapiens
SKGRSLIGKVDGTSHVTGKGV

SEQ ID NO: 43 - protein, 29 aa, Homo sapiens
LDPRSFLLRNPNDKYEPFWEDEEKNESGL

SEQ ID NO: 44 - protein, 28 aa, Homo sapiens
PAPRGYPGQVSANDSDTLELPDSSRALL